(19) United States
(12) Patent Application Publication
(10) Pub. No.: US 2021/0267524 A1
(43) Pub. Date: Sep. 2, 2021

(54) CONTACTLESS ELECTRODE FOR SENSING PHYSIOLOGICAL ELECTRICAL ACTIVITY

(71) Applicant: EasyG LLC, Beverly Hills, MI (US)

(72) Inventor: Alireza MOGHADDAMBAGHERI, Vancouver (CA)

(21) Appl. No.: 17/326,015

(22) Filed: May 20, 2021

Related U.S. Application Data
(63) Continuation of application No. PCT/US2019/063403, filed on Nov. 26, 2019.
(60) Provisional application No. 62/772,242, filed on Nov. 28, 2018.

(51) Int. Cl. A61B 5/30; A61B 5/273; A61B 5/00; A61B 5/316; A61B 5/268

(52) U.S. Cl. CPC A61B 5/30; A61B 5/273; A61B 5/0006; A61B 2560/04; A61B 5/268; A61B 2562/18; A61B 5/316

(57) ABSTRACT

Systems and apparatus for contactless measuring of biological electrical activity corresponding to an individual include an electrode capacitively coupled with a tissue surface of the individual and a high input impedance amplifier circuit for amplifying a sensing signal generated by the electrode. In some embodiments, the electrode comprises a sensing portion comprising a plurality of electrically conductive layers including a sensing layer, a guard layer and a grounding layer layered between electrically non-conductive insulating layers. Optionally, the sensing portion may also include an electrically conductive guard ring. The guard layer, guard ring and/or grounding layer may shield the sensing layer from external electromagnetic interference which may impinge on the sensing layer and/or maintain high input impedance of the high input impedance amplifier circuit. The high input impedance amplifier circuit may comprise an integrator circuit for biasing a high input impedance amplifier minimizing saturation of the high input impedance amplifier.

### CROSS-REFERENCE TO RELATED APPLICATIONS

This application is a continuation of Patent Cooperation Treaty (PCT) application No. PCT/US2019/063403 having an international filing date of 26 Nov. 2019 which in turn claims priority from, and the benefit under 35 U.S.C. § 119 in relation to, U.S. Application No. 62/772,242 filed 28 Nov. 2018. All of the applications in this paragraph are hereby incorporated herein by reference for all purposes.

### TECHNICAL FIELD

The technology described herein relates to electrodes for electrocardiography (ECG) systems, electroencephalography (EEG) systems, electromyography (EMG) systems, electrooculography (FOG) systems and/or similar systems, which detect physiological electrical activity at locations on, within, or proximate to, an individual's body.

### BACKGROUND

A conventional ECG system, for example, typically includes between 3 and 10 electrodes placed on areas of an individual's body to detect electrical activity of the individual's heart. The electrodes are connected to an ECG monitor by a commensurate number of wires/cables. A conventional ECG electrode typically includes a resistive sensor element. A conventional ECG is typically placed directly against the individual's skin, with possibly some conductive gel. A number of electrodes are placed against the individual's skin to detect the electrical characteristics of the heart (e.g. the current through or voltage across the resistive sensor element) at desired vantage points on the individual's body. The detected signals are relayed through the wires to the ECG monitor, which is typically located on a lab table or the like, away from the individual's body. A signal processing unit within the ECG monitor processes the signals to generate an ECG waveform which can be displayed on a display of the ECG monitor.

FIGS. 1 and 2 show three electrodes 10, 12, 14 arranged in the so-called Einthoven's triangle on an individual's body 16. As is known in the art, electrodes 10, 12 and 14 may be respectively referred to as the Right Arm (RA), Left Arm (LA) and Left Leg (LL) electrodes because of the locations that they are commonly placed on body 16. To generate an ECG signal, various potential differences are determined between the signals from electrodes 10, 12, 14. These potential differences are referred to as “leads”. Leads have polarity and associated directionality. The common leads associated with the Einthoven's triangle shown in FIGS. 1 and 2 include: lead I (where the signal from RA electrode 10 is subtracted from the signal from LA electrode 12); lead II (where the signal from RA electrode 10 is subtracted from the signal from LL electrode 14); and lead III (where the signal from LA electrode 12 is subtracted from the signal from LL electrode 14).

In addition to the leads shown in FIG. 2, other common leads associated with the Einthoven's triangle configuration include: the AVR lead (where one half of the sum of the signals from LA and LL electrodes 12, 14 is subtracted from the signal for RA electrode 10); the ACL lead (where one half of the sum of the signals from RA and LL electrodes 10, 14 is subtracted from the signal for LA electrode 12); and the AVF lead (where one half of the sum of the signals from RA and LA electrodes 10, 12 is subtracted from the signal for LL electrode 14). As is known in the art, the AVR lead is oriented generally orthogonally to lead III, the AVL lead is oriented generally orthogonally to lead II and the AVG lead is oriented generally orthogonally to lead I. The signals from each of these leads can be used to produce an ECG waveform 18 as shown in FIG. 3. Additional sensors can be added to provide different leads which may be used to obtain different views of the heart activity. For example, as is well known in the art, sensors for precordial leads V1, V2, V3, V4, V5, V6 may be added and such precordial leads may be determined to obtain the so-called 12 lead ECG.

Detected physiological electrical activity (e.g. electrical activity detected using, an ECG system, EEG system, EOG system, EMG system and/or the like) may, for example, be used to determine non-electrical physiological parameters, such as, for example a respiratory rate of a subject.

Some issues with traditional ECG technology make it an impediment for use, particularly in emergency response situations. The multiple electrodes and their corresponding wires may require extensive time to set up which may be critical in emergency circumstances. Having to maneuver around and detangle a large number of wires can be a nuisance. Multiple electrodes and wires can make it difficult to move an individual or administer medical aid to an individual. Further, it is almost impossible to connect electrodes and maneuver their corresponding wires in space-limited settings such as, for example, within the interior cabins of air planes, buses, cars, trucks, boats or the like. Signal noise from movement of the wires and wire tension can also degrade the quality of the ECG reading. Multiple wires can be particularly problematic during cardiac monitoring, where the ECG wires are attached to an individual for a long time. These issues with traditional ECG technology are exacerbated where there is a significant distance between the individual and the ECG monitor (i.e. where the electrode wires are long). EEG systems (which measure electrical activity of the brain), EMG systems (which measure electrical activity of skeletal and/or other muscles) and/or EOG systems (which measure electrical activity within the eye) may face similar problems.

In addition to the problems with wires, current ECG systems use contact electrodes. Such contact electrodes typically must be placed in direct contact with the individual's skin using an adhesive (e.g. conductive gels). The use of contact electrodes can be problematic in some circumstances. By way of non-limiting example, it may be undesirable or difficult to remove the individual's clothing in certain situations—e.g. where the individual may have privacy concerns, where the individual may have a condition which makes it undesirable or difficult to apply current-sensing electrodes to the skin—e.g. the individual is suffering from burns to the individual's skin, the individual has body hair which must be removed prior to using the contact electrodes, the individual is allergic and/or has a sensitivity to the adhesive, the individual is a prematurely born infant having sensitive and/or fragile skin or the like. The use of contact electrodes may also expose an individual to an electric shock hazard—e.g. failure of isolation circuitry isolating the contact electrodes from an electrical power system may result in electric shock of the individual. Also, EEG systems often require applying conductive gels between the sensor and the skin of the individual and/or abrasion of the individual's skin to create electrical contact between the sensor and the skin. It can take a long time (e.g. up to an hour or more) to apply the gel into EEG caps and/or nets that are used in EEG sensing systems. The gel used in EEG systems can diffuse through hair to create shorts between sensors and can dry out over time. Whether gel-coated or not, the caps or nets which hold EEG sensors in contact with skin can be uncomfortable for the individual being tested, making long term monitoring (e.g. a desire when evaluating certain conditions such as epilepsy) difficult.

There is a general desire for improved electrode systems for ECG, EEG, EMG and/or EOG systems. By way of non-limiting example, there is a general desire for an electrode that can provide greater flexibility for use by medical professionals and/or lay (non-medical) people in a variety of different circumstances. There is also a general desire for an electrode that may be more convenient and/or simple to use than existing contact electrodes. There is also a general desire for an improved electrode for detecting electrical activity in different locations on and/or within an individual's body, such as the heart (e.g. heart muscle), brain, the eyes, skeletal or other muscles, or the like.

The foregoing examples of the related art and limitations related thereto are intended to be illustrative and not exclusive. Other limitations of the related art will become apparent to those of skill in the art upon a reading of the specification and a study of the drawings.

### SUMMARY

The following embodiments and aspects thereof are described and illustrated in conjunction with systems, tools and methods which are meant to be exemplary and illustrative, not limiting in scope. In various embodiments, one or more of the above-described problems have been reduced or eliminated, while other embodiments are directed to other improvements.

One aspect of the invention provides a system for sensing biopotentials (i.e. physiological electrical activity) in an individual. The sensing system may be contactless. Such system comprises an electrode for generating a sensing signal indicative of a biopotential at a location of a body of the individual and a high input impedance amplifier circuit. The electrode comprises an electrically conductive sensing layer having a sensing surface, an opposing surface which opposes the sensing surface and one or more edge surfaces extending between the sensing surface and the opposing surface. The sensing surface is exposed for capacitive coupling to an outer tissue surface of the individual and is sensitive to electric field in a location and/or vicinity of the sensing surface. The electrode further comprises an electrically conductive guard layer proximate to the opposing surface of the sensing layer. The opposing surface of the sensing layer and the guard layer are separated by an electrically non-conductive layer. The guard layer electrically insulates the sensing layer from electromagnetic interference from electromagnetic energy that impinges on the guard layer. The guard layer may also prevent leakage current from flowing into the sensing layer (and thereby deteriorating the signal from the sensing layer and/or the high input impedance of the amplifier). The sensing layer is electrically coupled to an input of the high input impedance amplifier circuit to generate an amplifier output signal that depends at least in part on capacitive coupling between the sensing layer and the tissue surface of the individual.

In some embodiments, the electrode comprises an electrically conductive guard ring peripherally enclosing the sensing layer. An electrically non-conductive ring extending from an edge surface of the sensing layer to an inner edge surface of the guard ring separates the sensing layer from the guard ring. The guard ring electrically insulates the sensing layer from peripheral electromagnetic interference from peripheral electromagnetic energy that impinges on the guard ring. The guard layer may also prevent leakage current from flowing into the sensing layer (and thereby deteriorating the signal from the sensing layer and/or the high input impedance of the amplifier). In some embodiments, the high input impedance amplifier comprises a buffer amplifier for generating a buffered signal. In some embodiments, the buffered signal is electrically coupled to one or more of the guard layer and the guard ring. In some embodiments, the buffered signal is identical in amplitude and phase to the sensing signal. In some embodiments, the electrode comprises an electrically conductive grounding layer proximate to the guard layer and electrically coupled to a ground signal of the amplifier circuit for insulating one or more of the guard layer, the guard ring and the sensing layer from electromagnetic interference from electromagnetic energy that impinges on the grounding layer. An electrically non-conductive layer separates the grounding layer from the guard layer.

Another aspect of the invention provides a contactless method for sensing biopotentials in an individual, the method comprising the steps of using an electrode to generate a sensing signal indicative of a biopotential at a body location of the individual; and conditioning the generated sensing signal with a high input impedance amplifier circuit. The electrode used to generate the sensing signal comprises an electrically conductive sensing layer having a sensing surface, an opposing surface which opposes the sensing surface and one or more edge surfaces extending between the sensing surface and the opposing surface. The sensing surface is exposed for capacitive coupling to an outer tissue surface of the individual and is sensitive to electric field in a location and/or vicinity of the sensing surface. The electrode further comprises an electrically conductive guard layer proximate to the opposing surface of the sensing layer and is separated from the opposing surface by an electrically non-conductive layer. The guard layer electrically insulates the sensing layer from electromagnetic interference from electromagnetic energy that impinges on the guard layer.

Another aspect of the invention provides a contactless system for sensing biopotentials in an individual, the system comprising an electrode for generating a sensing signal indicative of a biopotential at a location of a body of the individual. The electrode comprises an electrically conductive sensing layer having a sensing surface, an opposing surface which opposes the sensing surface and one or more edge surfaces extending between the sensing surface and the opposing surface. The sensing surface is exposed for capacitive coupling to an outer tissue surface of the individual and is sensitive to electric field in a location and/or vicinity of the sensing surface. The system further comprises a high input impedance amplifier circuit. The sensing layer is electrically coupled to an input of the amplifier circuit to generate an amplifier output signal that depends at least in part on capacitive coupling between the sensing layer and the tissue surface of the individual. The system further comprises a biasing integrator circuit for maintaining at least a direct current component of the amplifier output signal within operational voltage limits of the amplifier circuit. The biasing circuit generates a biasing signal minimizing drift of the direct current component from a reference voltage. The biasing signal is electrically coupled to a biasing input of the amplifier circuit.

Another aspect of the invention provides a contactless method for sensing biopotentials in an individual, the method comprising the steps of capacitively coupling an electrode to an outer tissue surface of the individual to generate a sensing signal indicative of a biopotential at a location of the coupled outer tissue surface; conditioning the generated sensing signal with a high input impedance amplifier circuit; and using a biasing integrator circuit, maintaining at least a direct current component of the conditioned signal within operational voltage limits of the amplifier circuit. The biasing circuit generates a biasing signal minimizing drift of the direct current component from a reference voltage. The electrode comprises an electrically conductive sensing layer having a sensing surface, an opposing surface which opposes the sensing surface and one or more edge surfaces extending between the sensing surface and the opposing surface. The sensing surface is exposed for capacitive coupling to an outer tissue surface of the individual and is sensitive to electric field in a location and/or vicinity of the sensing surface. The biasing signal is electrically coupled to a biasing input of the amplifier circuit.

Another aspect of the invention provides a high input impedance amplifier circuit for receiving and conditioning a sensing signal generated by capacitively coupling the sensing surface of an electrode to an outer tissue surface of the individual. The amplifier circuit comprises a high impedance amplifier for receiving a generated sensing signal. A generated guard signal minimizes leakage currents from the sensing surface maintaining the amplifier circuit's high input impedance. In some embodiments, the amplifier circuit further comprises an integrator circuit for reducing and/or minimizing voltage drifts that may result in saturation of the high impedance amplifier.

In addition to the exemplary aspects and embodiments described above, further aspects and embodiments will become apparent by reference to the drawings and by study of the following detailed descriptions.

### BRIEF DESCRIPTION OF THE DRAWINGS

Exemplary embodiments are illustrated in referenced figures of the drawings. It is intended that the embodiments and figures disclosed herein are to be considered illustrative rather than restrictive.

FIG. 1 is a schematic illustration of the electrodes of a conventional ECG system arranged on the individual's body in an Einthoven's triangle configuration.

FIG. 2 is a schematic illustration of the electrodes of a conventional ECG system arranged in an Einthoven's triangle configuration and a number of corresponding leads.

FIG. 3 is a typical ECG waveform of the type that might be displayed on an ECG system.

FIG. 4 is a perspective view of an example contactless electrode system according to an exemplary embodiment.

FIG. 4A is a plan view of the electrode of the FIG. 4 electrode system.

FIG. 5 is a cross-sectional view of an example embodiment of a sensing portion of the FIG. 4 electrode.

FIGS. 5A-5B are cross-sectional views of example alternative embodiments of a sensing portion of the FIG. 4 electrode.

FIG. 6A is a block diagram of an example wired embodiment a contactless electrode system.

FIG. 6B is a block diagram of an example wireless embodiment of a contactless electrode system.

FIG. 6C is a block diagram of an example multi-sensor embodiment of a contactless electrode system.

FIG. 7 is an electrical schematic illustration of an amplifier circuit suitable for use with the FIG. 4 electrode system according to an exemplary embodiment.

FIGS. 7A-D are electrical schematic illustrations various example embodiments of an amplifier circuit suitable for use with the FIG. 4 electrode system.

FIG. 8A is a graph depicting simulation results which show undesirable saturation in an amplifier circuit. FIG. 8B is a graph depicting simulation results of an amplifier circuit comprising an integrator circuit to mitigate the saturation issue in FIG. 8A.

FIG. 9 is a schematic illustration of making biopotential measurements using a current sense amplifier and two contactless electrodes.

FIG. 10 is a schematic illustration of an example ECG system incorporating the FIG. 4 electrode system.

FIG. 11 is a perspective view of an example portable device incorporating the FIG. 4 electrode system.

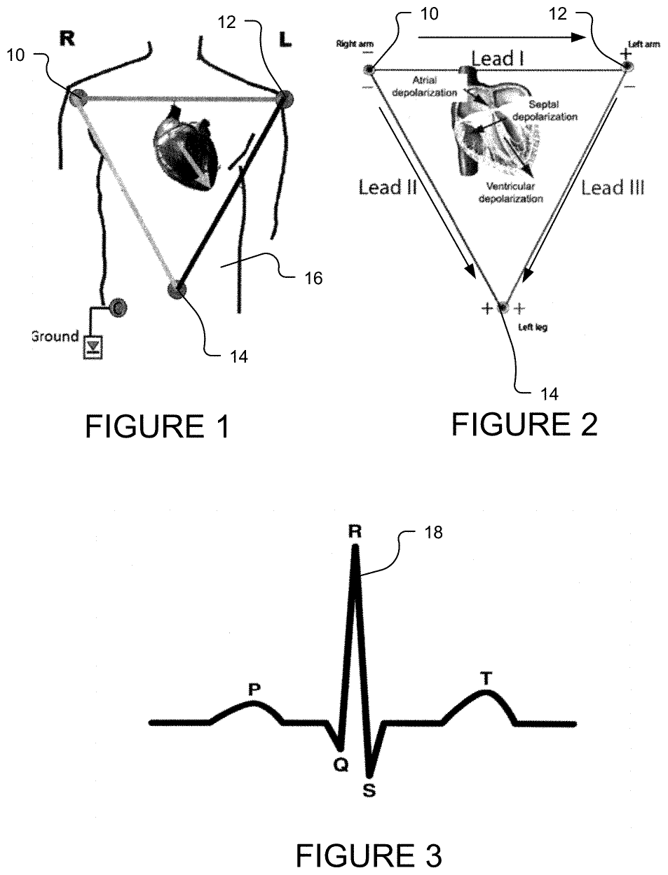

### DESCRIPTION

Throughout the following description specific details are set forth in order to provide a more thorough understanding to persons skilled in the art. However, well known elements may not have been shown or described in detail to avoid unnecessarily obscuring the disclosure. Accordingly, the description and drawings are to be regarded in an illustrative, rather than a restrictive, sense.

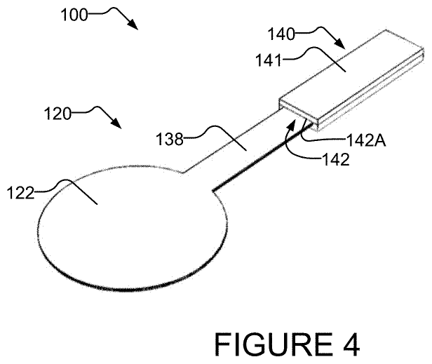

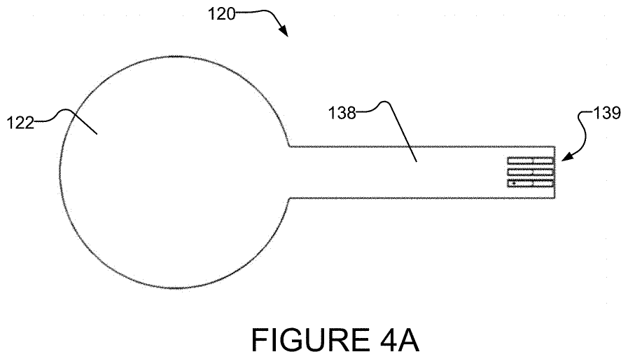

FIG. 4 illustrates a perspective view of an electrode system 100 according to an example embodiment. Electrode system 100 of the FIG. 4 embodiment comprises a contactless electrode 120 (e.g. an electrode capable of sensing biopotentials of a subject without contacting the body of the subject) removably coupled to a housing 141 enclosing an amplifier circuit 140. In some embodiments, amplifier circuit 140 has high input impedance to avoid loading contactless electrode 120. In some embodiments, it is not necessary that electrode system 100 be contactless and a contact-based electrode (not shown) may be provided in addition to, or in the alternative to, contactless electrode 120. Contactless electrode 120 is electrically connected to amplifier circuit 140 through suitable electrical wirings, leads, pins, pads or the like.

Housing 141 may, for example, be fabricated from electrically conductive material and may electrically shield (i.e. isolate) amplifier circuit 140 from external undesired electromagnetic interference from electromagnetic energy that impinges on housing 141. In some embodiments, housing 141 may be hermetically sealed. Hermetic sealing of housing 141 prevents moisture, dust, debris or the like from entering housing 141 and possibly having a deleterious impact on the input impedance or other performance characteristics of amplifier circuit 140. Housing 141 may, for example, be sealed using an epoxy resin or the like.

In the FIG. 4 example embodiment, contactless electrode 120 comprises a sensing portion 122 and a projection member 138. Projection member 138 may extend outwardly from sensing portion 122 of electrode 120. The length of projection member 138 may be longer than that of sensing portion 122. Projection member 138 may be flexible. Projection member 138 may include electrically conductive contact plates 139 (see FIG. 4A) on a distal end of projection member 138. Sensing portion 122 of electrode 120 may, for example, be electrically coupled (e.g. routed) to contact plates 139 via electrical leads and/or contacts (not explicitly shown), as described herein.

Projection member 138 may be connected to housing 141 through a suitably configured electrical connection port 142 of housing 141. Connection port 142 may comprise electrically conductive contact plates 142A engageable with contact plates 139 of electrode 120. Projection member 138 may be mechanically coupled to connection port 142 to electrically engage contact plates 139 and 142A to electrically couple electrode 120 with amplifier circuit 140. Contact plates 139 and 142A may be fabricated from gold, silver, copper, other conductive metals, combinations thereof and/or the like.

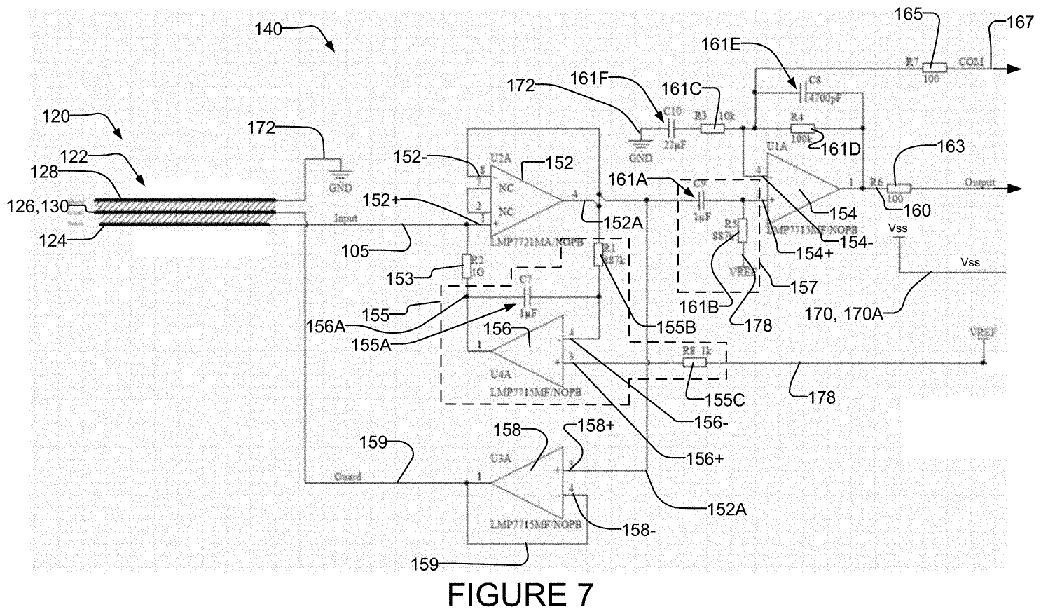

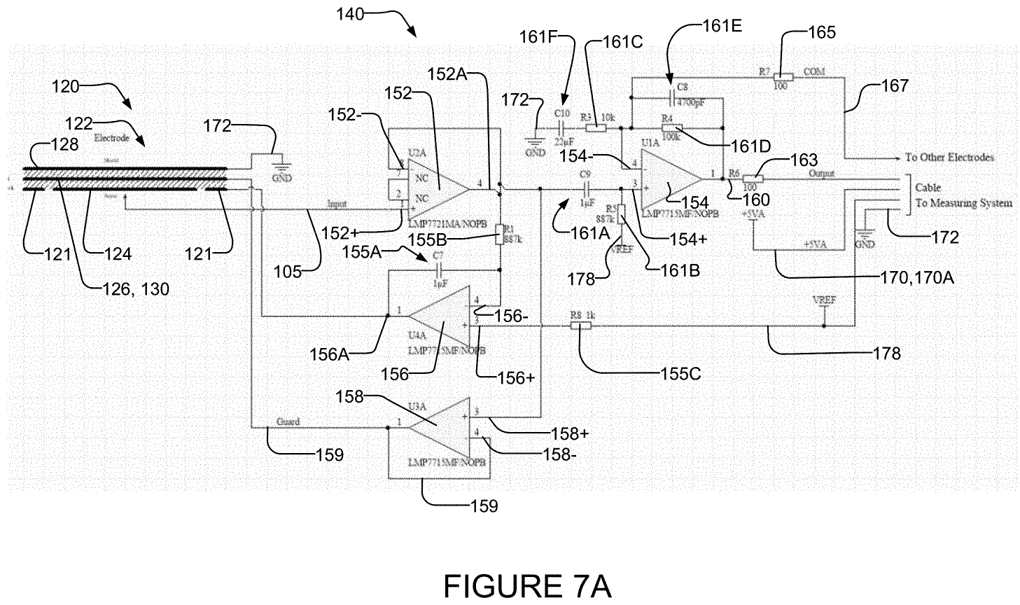

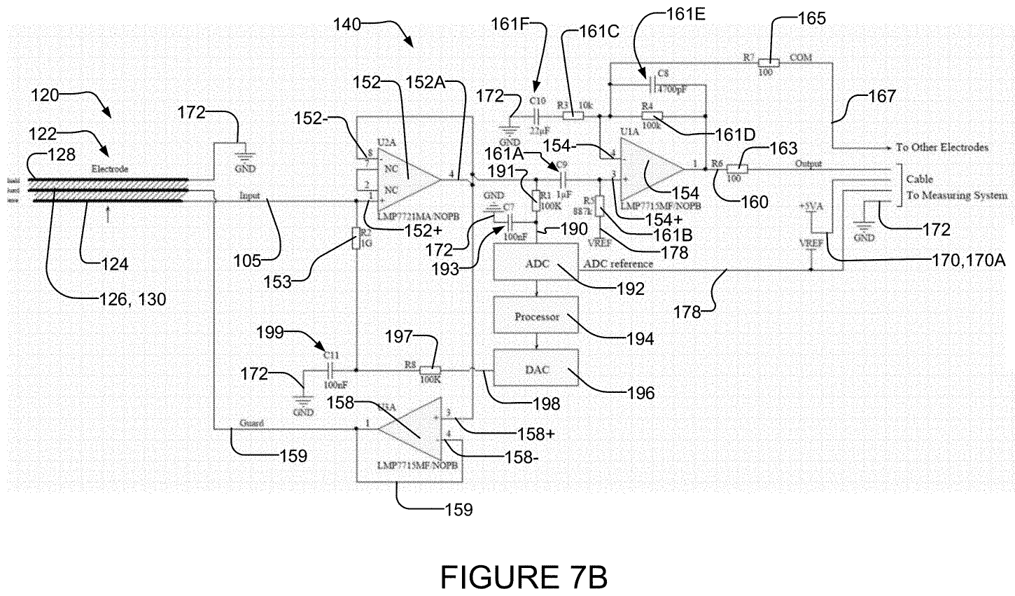

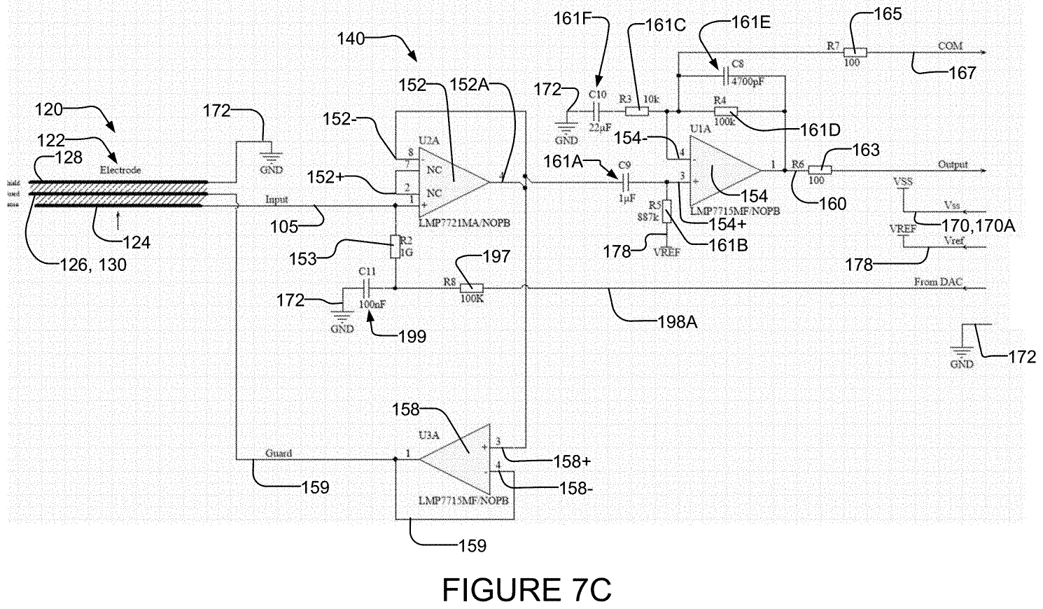

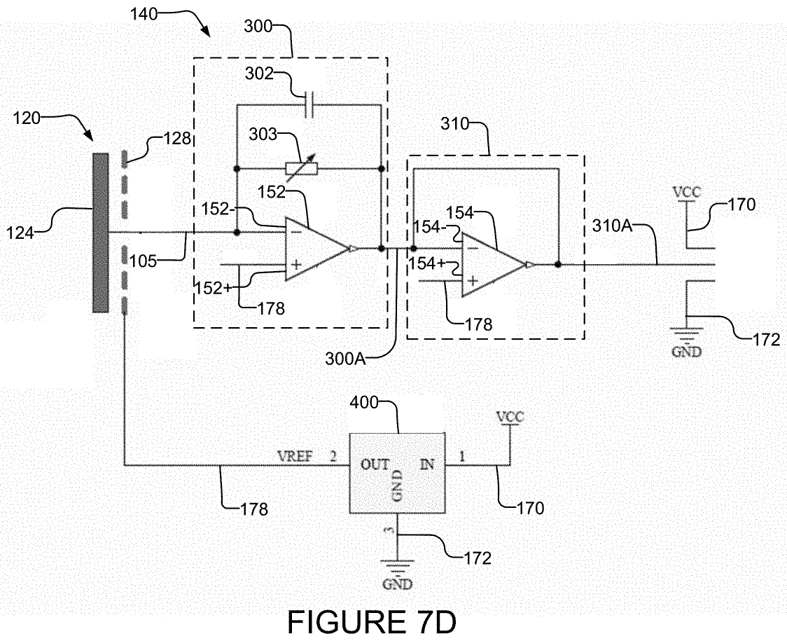

Sensing portion 122 may be brought into proximity of the body (e.g. skin) of an individual, where sensing portion 122 may be capacitively coupled to the individual's outer tissue surface, so that sensing portion 122 is sensitive to (e.g. exhibits a capacitance or otherwise generates an electrical signal that depends on) electric field in a location and/or vicinity thereof. Sensing portion 122 may, for example, be capacitively coupled to anterior (i.e. front) and/or posterior (i.e. rear) outer tissue surfaces of the individual. More specifically, placement of sensing portion 122 in proximity to an outer tissue surface of the individual exposes sensing portion 122 to one or more electric fields corresponding to one or more measurable biopotentials (e.g. electrical activity within the individual). Exposure of sensing portion 122 to such electric field causes sensing portion 122 to exhibit an electrical sensing signal 105 (FIG. 7) representative, at least in part, of the electric field to which sensing portion 122 was exposed. Advantageously, physical contact between sensing portion 122 and an outer tissue surface is not required for generation of sensing signal 105. In some embodiments, electrode 120 may measure biopotentials within an individual without removal of the individual's clothing.

In the FIG. 4 example embodiment, sensing portion 122 is circular, although this is not necessary. A surface area of sensing portion 122 may be varied, for example, depending on biopotentials to be measured and/or physical size constraints (e.g. EEG requires many electrodes in close vicinity of one another). For example, a diameter of 50 mm (or surface area of ˜2000 mm²) may be suitable for measurement of biopotentials for an ECG system. A diameter of 20 mm (or a surface area of ˜315 mm²) may, for example, be suitable for measurement of biopotentials for an EEG or EMG system. The sensing surface of sensing portion 122 may be triangular, elliptical, rectangular, trapezoidal, hexagonal, octagonal or any other suitable shape. In some embodiments, electrode 120 is flexible allowing electrode 120 and sensing portion 122 to adapt and/or conform to body contours which may be individual specific.

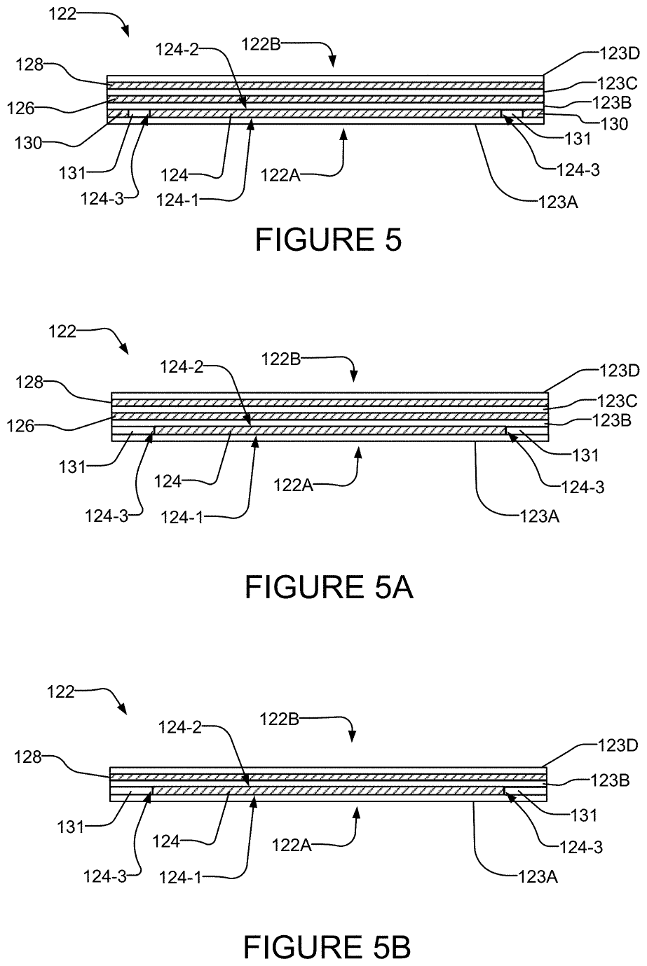

FIG. 5 illustrates a vertical cross-sectional view of sensing portion 122 of the FIG. 4 electrode 120 according to an example embodiment. Sensing portion 122, as illustrated, is multi-layered comprising a plurality of electrically conductive layers 124, 126, 128 (shown as hatched in FIG. 5) and an electrically conductive ring 130 (also shown as hatched) layered in between electrically non-conductive insulating layers 123A, 123B, 123C, 123D (collectively insulating layers 123—shown as white in FIG. 5) and an electrically insulating ring 131 (also shown as white).

In some embodiments, electrically conducting ring 130 is concentric with electrically insulating ring 131. In some embodiments, electrically insulating ring 131 is enclosed by electrically conducting ring 130. In some embodiments, an inner circumference of electrically conducting ring 130 contacts an outer circumference of electrically insulating ring 131. In some embodiments, electrically conducting ring 130 and electrically insulating ring 131 are located in a second layer of the plurality of layers of sensing portion 122.

In the FIG. 5 embodiment conductive layer 124 provides a sensing layer. Sensing layer 124, which is proximate to a sensing side 122A of sensing portion 122, is sensitive to electric field associated with biopotentials. In some embodiments, sensing layer 124 is located in the second layer comprising the electrically conducting ring 130 and the electrically insulating ring 131 described above.

Sensing layer 124 may be described as an antenna for receiving one or more electric fields corresponding to electrical activity within the body of the individual. One or more electric fields associated with biopotentials from the individual excites electrons within sensing layer 124 generating sensing signal 105 representative of the biopotentials. Electrical lead(s) or contact(s) (not explicitly shown) within electrode 120 may, for example, electrically couple sensing layer 124 to contact plates 139 described herein. Insulating layer 123A extending across an outer sensing surface 124-1 of sensing layer 124 protects sensing layer 124 from exposure to external environmental elements such as, for example, moisture, dust, bodily fluids or the like. Insulating layer 123A, in combination with insulating layer 123D, also facilitates disinfection and/or cleaning of sensing side 122A and non-sensing side 122B of sensing portion 122.

External electromagnetic interference, such as, for example, from cellular phones, Wi-Fi routers, Bluetooth coupled devices, cordless telephones, inductive chargers, multimedia displays, power lines and/or the like may reduce a signal-to-noise ratio (SNR) of sensing signal 105. In particular embodiments, as shown in FIG. 5, for example, sensing portion 122 comprises one or more of electrically conductive elements which may include: guard layer 126, guard ring 130 and/or grounding layer 128. In such embodiments, guard layer 126 may extend across an opposing surface 124-2 of sensing layer 124 (i.e. a surface 124-2 opposing sensing surface 124-1). Guard ring 130 may peripherally enclose edge surfaces 124-3 of sensing layer 124. Insulating layer 123B and/or insulating ring 131 may be located between guard layer 126 and guard ring 130 to electrically isolate guard layer 126 and guard ring 130 from sensing layer 124. Electrical lead(s) or contact(s) (not explicitly shown) within electrode 120 may, for example, electrically couple guard layer 126 and/or guard ring 130 to contact plates 139 described herein.

In some embodiments, guard layer 126 and/or guard ring 130 are electrically coupled to amplifier circuit 140 to receive buffer signal 159 from amplifier 158, as described elsewhere herein (see discussion of FIG. 7). Buffer signal 159, as described herein, may be similar or substantially identical in amplitude and phase to sensing signal 105 generated from sensing layer 124 (in preferred embodiments buffer signal 105 is identical to sensing signal 105). In such embodiments, continuously receiving buffer signal 159 at guard layer 126 and/or guard ring 130 equalizes electric potentials across sensing layer 124 and guard layer 126 and/or guard ring 130, thereby preventing extraneous current flow between sensing layer 124 and guard layer 126 and/or guard ring 130. In this manner, sensing layer 124 may be actively electrically shielded from external electromagnetic interference from electromagnetic energy which impinges on guard layer 126 and/or guard ring 130 and/or from leakage currents escaping sensing layer 124. Preventing extraneous current flow to and/or from sensing layer 124 desirably maintains a high input impedance of amplifier circuit 140. Guard layer 126 and guard ring 130 may, for example, reduce leakage currents from sensing layer 124 and/or impingement of external electromagnetic interference on sensing layer 124, thereby maintaining the high input impedance of amplifier circuit 140, even in embodiments where buffer signal 159 is not entirely identical in amplitude and phase to sensing signal 105.

Grounding layer 128 of the illustrated embodiment is proximate to non-sensitive side 122B of sensing portion 122 (alternatively, grounding layer 128 may be referred to as “shielding layer 128”). Grounding layer 128 may further improve the SNR of sensing signal 105 by, for example, further minimizing effects of external electromagnetic interference on sensing layer 124, guard layer 126 and/or guard ring 130.

Grounding layer 128 is electrically coupled to ground signal 172 of amplifier circuit 140. Impingement of external electromagnetic interference on grounding layer 128 electrically grounds any impinging electromagnetic interference precluding such electromagnetic interference from impinging sensing layer 124, guard layer 126 and/or guard ring 130. Insulating layer 123C extending across an internal surface of grounding layer 128 electrically isolates grounding layer 128 from guard layer 126. Insulating layer 123D extending across an exterior surface of grounding layer 128 protects grounding layer 128 from exposure to external environmental elements such as, for example, moisture, dust, bodily fluids or the like. Electrical lead(s) and/or contact(s) (not explicitly shown) within electrode 120 may, for example, electrically couple grounding layer 128 to contact plates 139 described herein.

Electrically conductive layers 124, 126, 128 and electrically conductive ring 130 (i.e. sensing layer 124, guard layer 126, grounding layer 128 and guard ring 130 respectively) may, for example, be fabricated from or otherwise comprises any suitable electrically conductive material such as gold, silver, copper, other conductive metals, electrically conductive polymer, combinations thereof and/or the like. In some embodiments, electrically conductive layers 124, 126, 128 and electrically conductive ring 130 may, for example, be fabricated from a uniform material. In other embodiments, electrically conductive layers 124, 126, 128 and electrically conductive ring 130 may be fabricated from different materials. In some embodiments, electrically conductive layers 124, 126, 128 and/or electrically conductive ring 130 are flexible. This may be the case, for example, when such layers are fabricated from electrically conductive polymers.

Electrically insulating layers 123 and electrically insulating ring 131 are electrically non-conductive. For example, insulating layers 123 and insulating ring 131 may comprise one or more dielectric materials, such as polyethylene, polyimide, polypropylene, other suitable plastics and/or the like. In some embodiments, electrically insulating layers 123 and electrically insulating ring 131 may be fabricated from a uniform material. In other embodiments, electrically insulating layers 123 and electrically insulating ring 131 may be fabricated from different materials. In some embodiments, electrically insulating layers 123 and electrically insulating ring 131 are flexible.

In some embodiments, guard ring 130 may be excluded (e.g. removed) from sensing portion 122 of electrode 120, as illustrated in FIG. 5A. In such embodiments, insulating ring 131 peripherally encloses outer edge surfaces 124-3 of sensing layer 124 and extends outwards from an outer edge surface 124-3 of sensing layer 124 to an outer edge surface of sensing portion 122 of electrode 120. Such embodiments, may be preferable, for example where the dimensions (e.g. the thickness) of outer edge surface(s) 124-3 is relatively small.

In some embodiments, guard layer 126 may be excluded (e.g. removed) from sensing portion 122 of electrode 120, as illustrated in FIG. 5B. Such embodiments, may be preferable, for example where it is desirable to minimize the number of components of electrode 120 and/or amplifier circuit 140.

In some embodiments, electrode 120 is flexible (i.e. one or more components of electrode 120 are flexible). In some such embodiments, electrode 120 may, for example, conform to one or more body contours of the individual. For electrode 120 to be flexible, electrically conductive layers 124, 126, 128, electrically conductive ring 130 and/or contact plates 139 may be mounted onto flexible PCBs. Each flexible PCB may, for example, comprise a flexible plastic substrate (e.g. a substrate fabricated from polyimide, conductive polyester film or other suitable flexible plastic material), PEEK or the like. In some embodiments, multiple conductive components may be mounted on a single flexible PCB (e.g. mounting contact plates on a single flexible PCB). In some embodiments, a flexible PCB may comprise multiple conductive components mounted on different electrically isolated layers of the PCB (e.g. mounting electrically conductive layers 124, 126, 128 on different electrically isolated layers of a single flexible PCB).

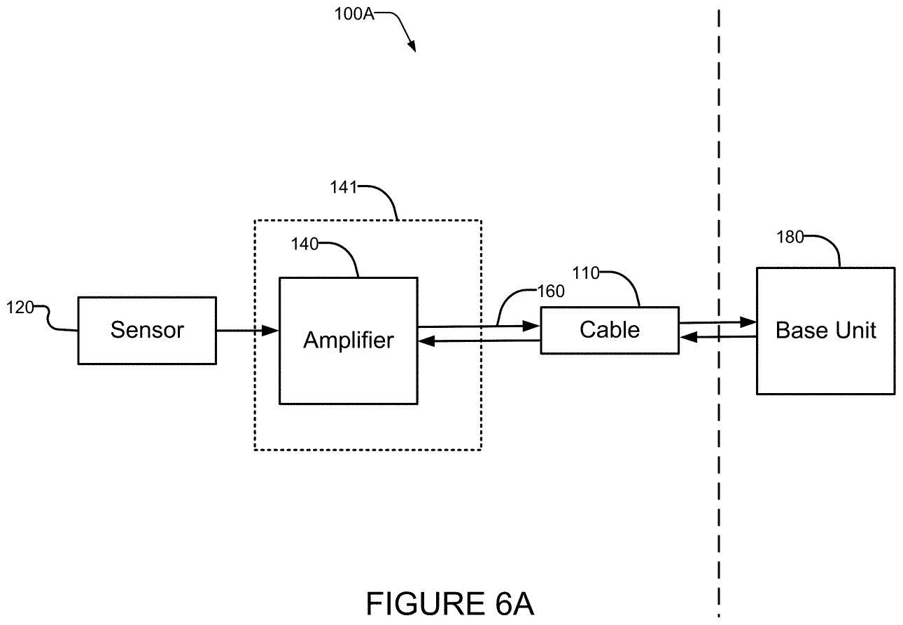

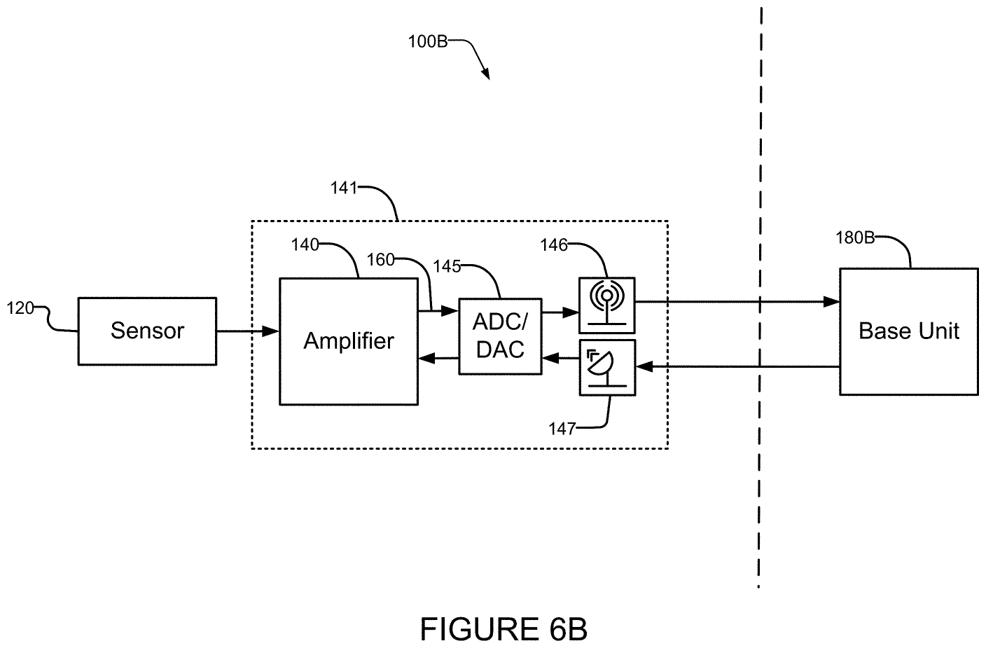

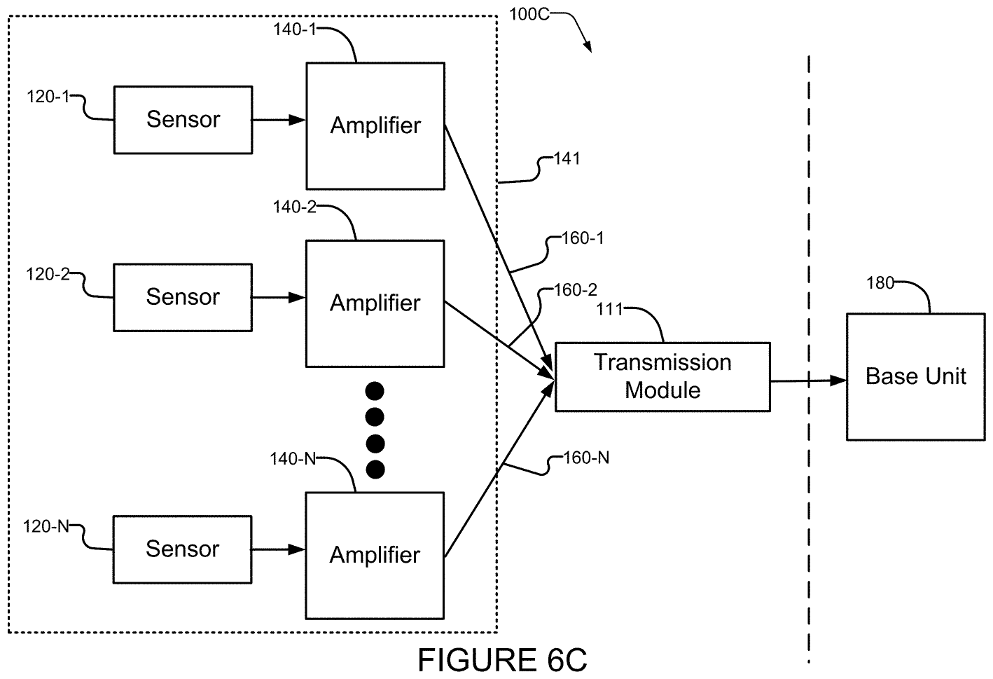

FIG. 6A is a block diagram of an example wired embodiment of contactless electrode system 100A. Contactless electrode system 100A comprises a cable 110. Cable 110 electrically couples amplifier circuit 140 to a base unit 180. Base unit 180 may comprise suitable hardware for receiving and processing amplified signal 160 from amplifier circuit 140. Examples of suitable hardware include, but are not limited to: analogue to digital converters, processors, controllers, displays, etc.

In a currently preferred embodiment, cable 110 is bidirectional electrically. Cable 110 may transmit an amplified signal 160 from amplifier circuit 140 to base unit 180 concurrently with transmitting a signal at the level of power supply 170 and a ground signal 172 which supply electrical power 170 and a ground reference 172 from base unit 180 to amplifier circuit 140.

Contactless electrode system 100A may be the preferred embodiment for use in hospital settings. For example, contactless electrode system 100A may be especially suitable for use in the intensive care unit (ICU), intensive therapy unit (ITU), Neonatal Intensive Care Unit (NICU) and/or critical care unit (CCU) of a hospital.

FIG. 6B is a block diagram of an example wireless embodiment of contactless electrode system 101B. Contactless electrode system 100B digitizes amplified signal 160 on board before transmitting a corresponding signal to base unit 180. Contactless electrode system 100B may transmit data to and/or receive data from base unit 180 wirelessly. Contactless electrode system 101B comprises suitable analogue to digital converters 145, transmitters 146, and receivers 147 for communicating signals wirelessly between amplifier circuit 140 and base unit 180.

By transmitting amplified signal 160 to base unit 180 wirelessly, contactless electrode system 100B may be adapted for use in non-hospital settings, as described elsewhere herein. For example, contactless electrode system 100B may be installed in cars, beds, cellphone cases, clothing, etc. Contactless electrode system 100B may transmit and/or receive signals from a wide range of base units 180B including, but not limited to: mobile phones, smart watches, personal computers, engine control modules (in a car), etc.

In some embodiments, housing 141 encloses amplifier circuit 140, digital converters 145, transmitters 146, and receivers 147. In some embodiments, some or all of amplifier circuit 140, digital converters 145, transmitters 146, and receivers 147 are fabricated on the same circuit board.

FIG. 6C is a block diagram of a contactless electrode system 100C according to an example embodiment. Contactless electrode system 100C comprises a plurality of electrodes 120-1, 120-2, . . . , 120-N electrically coupled to a plurality of corresponding amplifier circuits 140-1, 140-2, . . . , 140-N. A transmission module 111 transmits amplified signals 160-1, 160-2, . . . , 160-N from amplifier circuits 140-1, 140-2, . . . , 140-N to base unit 180. In some embodiments, transmission module 111 comprises one or a plurality of cables 110. In some embodiments, transmission module 111 comprises suitable analogue to digital converters 145, transmitters 146, and receivers 147 for transmitting signals wirelessly to base unit 180.

In the example embodiment shown in FIG. 6C, electrodes 120-1, 120-2, . . . , 120-N and their corresponding amplifier circuits 140-1, 140-2, . . . , 140-N are contained in the same housing 141 although this is not necessary. In some embodiments, electrodes 120-1, 120-2, . . . , 120-N and their corresponding amplifier circuits 140-1, 140-2, . . . , 140-N are fabricated on the same printed circuit board. In some embodiments, electrodes 120-1, 120-2, . . . , 120-N and their corresponding amplifier circuits 140-1, 140-2, . . . , 140-N are built as a single pad. The pad may be made of a suitably flexible material (e.g. silicone) which facilitates easy sanitization.

Contactless electrode system 100C may optionally receive signals such as control signals and/or power signals from base unit 180. Contactless electrode system 100C may optionally incorporate suitable sensor selection algorithms. The sensor selection algorithms may advantageously make contactless electrode system 100C adaptable for patients with different body sizes and may also achieve optimal ECG signals by selecting sensors to minimize or mitigate noise, artifacts and/or the like and/or to maximize signal amplitude.

In some embodiments (e.g. see FIGS. 6A-6C), transmitting an amplified signal 160 to base unit 180 advantageously improves signal to noise ratio.

In some embodiments, housing 141 comprises suitable silicone materials which may advantageously allow for improved sanitization of housing 141. In some embodiments, housing 141 comprises suitable fabric materials which may advantageously allow system 100 to be worn by a patient (e.g. as part of clothing). In some embodiments, housing 141 is made of suitable waterproof materials such that housing 141 with its enclosed components (e.g. electrodes 120, amplifier circuits 140, etc.) is washable. In some embodiments, housing 141 is made of suitable fireproof materials. In some embodiments, housing 141 is made of suitable materials that are flexible.

FIG. 7 schematically shows an amplifier circuit 140 suitable for use with electrode 122 according to an example embodiment.

Amplifier circuit 140 comprises a first amplifier 152 for receiving sensing signal 105 from sensing portion 122 of electrode 120 (e.g. from sensing layer 124). In the FIG. 7 illustrated embodiment, sensing signal 105 is input to amplifier 152 by, for example, electrically coupling sensing layer 124 to non-inverting input 152+ of amplifier 152 using any method described herein or known in the art. Amplifier 152 is configured as a unity gain amplifier (i.e. an amplifier having a voltage gain of 1) by feeding back output signal 152A to the inverting input 152− of amplifier 152 in the FIG. 7 example embodiment, although this is not necessary.

To maximize the sensitivity of amplifier circuit 140 to sensing signal 105, amplifier 152 may, for example, be a high input impedance amplifier. High input impedance of amplifier 152 (as a result of amplifier 152 having a low bias current) reduces electrical loading of sensing signal 105. Reducing electrical loading of sensing signal 105 can advantageously allow amplifier circuit 140 to receive a larger sensing signal 105 (compared to a smaller sensing signal 105 if amplifier 152 did not have a high input impedance).

In some embodiments, amplifier 152 may, for example, be a low input bias current operational amplifier. In some embodiments, amplifier 152 may comprise a low input bias current operational amplifier manufactured by Texas Instruments of Dallas, Tex. under part number LMP7721 or the like. In some embodiments, amplifier 152 has a minimum specified input bias current of 3 fA (i.e. 3 femtoamperes). In some embodiments, amplifier 152 has a maximum specified input bias current of 90 fA (at 85° C.).

Second amplifier 154 of amplifier circuit 140 may amplify output signal 152A of first amplifier 152, thereby generating amplified signal 160. Output signal 152A may, for example, be input into amplifier 154 at non-inverting input 154+ of amplifier 154.

In some embodiments, as illustrated in FIG. 7, output signal 152A may first pass through a high-pass filter 157 (e.g. a first degree high-pass filter, as is the case of the illustrated embodiment, or some higher order filter). High-pass filter 157 may comprise, for example, a capacitor 161A and a resistor 161B, as is the case with the illustrated embodiment of FIG. 7. Capacitor 161A electrically couples output 152A of first amplifier 152 to non-inverting input 154+ of second amplifier 154. Resistor 161B electrically couples reference voltage 178 to non-inverting input 154+. Varying capacitance of capacitor 161A and/or resistance of resistor 161B varies a frequency response of high-pass filter 157. In some embodiments, capacitor 161A and resistor 161B may, for example, have a capacitance of 1 ρF and a resistance of 887 kΩ respectively. Increasing the capacitance of capacitor 161A and/or the resistance of resistor 161B raises a cut-off frequency of high-pass filter 157. Conversely, decreasing the capacitance of capacitor 161A and/or the resistance of resistor 161B lowers the cut-off frequency of high-pass filter 157. An ideal frequency response of high-pass filter 157 may be different for different biopotentials (e.g. ECG vs EEG, ECG vs EMG, etc.). High-pass filter 157 may advantageously eliminate low frequency noise corresponding to slow motion artifacts, such as the acceleration or deceleration of a vehicle in a vehicular application.

Amplifier 154 may be suitably arranged and electrically connected to suitable resistors and/or capacitors to control its gain, corner low frequency, corner high frequency, etc. In the example embodiment shown in FIG. 7, resistor 161B may bias amplifier 154 by setting a steady state output voltage of amplifier 154 to a vicinity of a specific voltage. Resistor 161B may also, in combination with resistors 161C, 161D and capacitor 161F, form a gain stage of amplifier 154. Voltage gain of amplifier 154 may be varied by varying resistances of one or more of resistors 161C, 161D. Frequency response of amplifier 154 may be varied by varying resistance of one or more of resistors 161C, 161D and/or varying capacitance of one or more of capacitors 161E, 161F. Resistor 161C and capacitor 161F (electrically coupled in series as shown in FIG. 7) may set a corner low frequency of amplifier 154. Resistor 161D and capacitor 161E (electrically coupled in parallel as shown in FIG. 7) may set a corner high frequency of amplifier 154. For example, decreasing the resistance of resistor 161D and/or capacitance of capacitor 161E may increase the corner high frequency of amplifier 154 (i.e. increases the frequency response bandwidth of amplifier 154). In some embodiments, amplifier 154 has a voltage gain in a range of 2-20. In some embodiments, this range is 5-15. In some embodiments, this range is 6-12. Other levels of gain are possible.

It may be desirable for amplifier circuit 140 to use different corner high (i.e. cut-off) frequencies for different applications (e.g. ECG vs. EEG, EEG vs. EMG, etc.). In some embodiments, resistors 161C, 161D may have resistances of 10 kΩ and 100 kΩ respectively and capacitors 161E, 161F may have capacitances of 4,700 ρF and 22 ρF respectively. In some such embodiments, amplifier 154 has a voltage gain of 11 (1+R4/R3), a corner low frequency of 0.72 Hz and a corner high frequency of 338.62 Hz. In some embodiments (e.g. some EEG systems), resistors 161C, 161D may have resistances of 5 kΩ and 50 kΩ respectively or capacitors 161E, 161F may have capacitances of 2350 μF and 11 μF respectively. Other values of resistances, capacitances, gains and corner high frequencies may be used.

In some embodiments, one or more of passive electrical components 161 (e.g. resistors 161B, 161C, 161D and/or capacitors 161A, 161E, 161F) may, for example, be tunable (i.e. resistance and/or capacitance values may be varied) in real-time and/or in a calibration context, thereby varying voltage gain and/or frequency response of amplifier 154 in real-time and/or in a calibration context. Real-time tuning and/or pre-use calibration of amplifier 154 may, for example, generate an amplified signal 160 optimized for a use-specific purpose (e.g. ECG specific, EEG specific, etc).

Amplified signal 160 output from amplifier 154 may, for example, be transmitted to base unit 180 using cable 110 (FIG. 4) as described herein. Optionally, amplified signal 160 may pass through resistor 163 prior to being received by cable 110. Optional resistor 163 may electrically safeguard amplifier 154 and/or amplifier circuit 140 from adverse electrical events such as, for example, high capacitive loading of cable 110 and/or base unit 180, an electrical short circuit within cable 110 or the like. In some embodiments, resistor 163 may have a resistance in the range of 100 to 1000.

Amplifier circuit 140 may further comprise a buffer amplifier 158. Buffer amplifier 158 may be used to generate a buffer signal 159 receivable by guard layer 126 and/or guard ring 130 (if guard ring 130 is included) of sensing portion 122 of electrode 120. As described herein, buffer signal 159 may be used to reduce adverse impacts of external electromagnetic interference and/or leakage currents on sensing plate 124 and/or maintain the high-input impedance of amplifier circuit 140. In preferred embodiments, output signal 159 is similar and substantially identical in amplitude and phase to sensing signal 105. In such embodiments, output signal 152A of amplifier 152 is input to buffer amplifier 158 at non-inverting input 158+ of buffer amplifier 158. Amplifier 158 is configured as a unity gain amplifier (i.e. having a voltage gain of 1) by directly feeding buffer signal 159 back to buffer amplifier 158 at inverting input 158− in the FIG. 7 example embodiment, although this is not necessary.

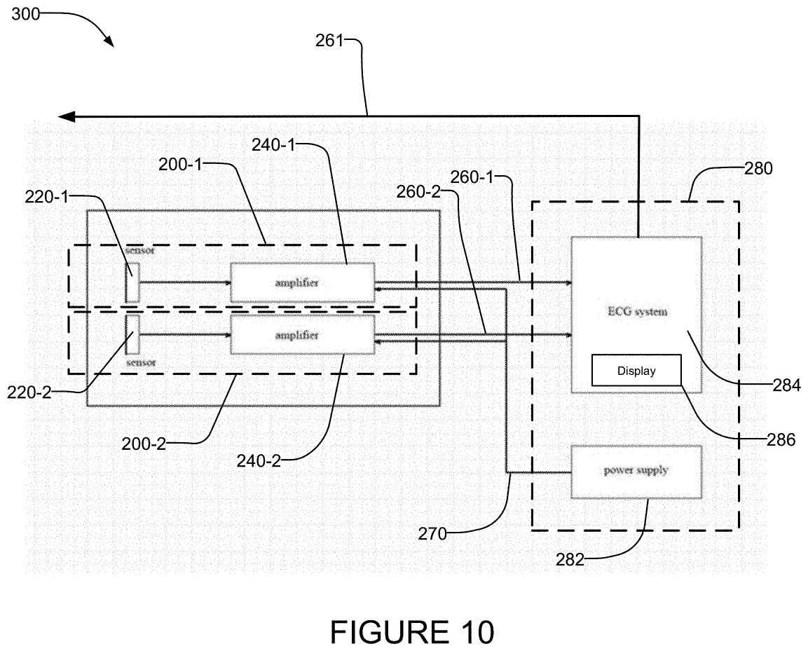

In some embodiments, amplifier circuit 140 comprises optional resistor 165 which samples inverting input 154− of second amplifier 154 to generate a “COM” signal 167. Resistor 165 may, for example, have a resistance in the range of 470 to 1000. In biopotential measurement systems comprising multiple electrode systems 100 (such as, for example, in the embodiment shown in FIG. 6C and FIG. 10), a plurality of “COM” signals 167 may be combined to generate a common mode node. In such embodiments, the generated common mode node may be used, for example, for common mode interference rejection (e.g. rejection of external electromagnetic interference received by an individual's body as a result of the body acting as an antenna or otherwise common to multiple electrodes). Common mode interference rejection may be implemented by capacitively feeding back the common mode node to an individual's body using one or more of electrode systems 100. This approach may, for example, be termed “Right Leg Drive” (RLD) and/or “Driven Right Leg” (DRL). “COM” signal 167 may, for example, be transmitted from amplifier circuit 140 to base unit 180 using cable 110. In some embodiments, “COM” signal 167 is wirelessly transmitted from amplifier circuit 140 to base unit 180 using a suitable wireless communication interface.

In some embodiments, amplifier circuit 140 comprises a resistor 153 to bias non-inverting input 152+ of amplifier 152 (i.e. the input impedance of amplifier circuit 140 is dependent at least in part on resistor 153). In some embodiments, input impedance of amplifier circuit 140 is equivalent to a resistance value of resistor 153 (i.e. in embodiments where the resistance value of resistor 153 is small (e.g. 10 GΩ) when compared to an input impedance of amplifier 152). In other embodiments, input impedance of amplifier circuit 140 is equivalent to a total resistance value of resistor 153 in parallel with the input impedance of amplifier 152.

Varying input impedance of amplifier circuit 140 may, for example, vary sensitivity of contactless electrode system 100. In such embodiments, a desired use-specific (e.g. ECG specific, EEG specific, etc.) sensitivity may be set by varying resistance of resistor 153. Sensitivity of amplifier circuit 140 to sensing signal 105 may be increased or decreased by increasing or decreasing resistance of resistor 153 respectively. For example, a resistance value of resistor 153 between 1 GΩ and 10 GΩ may be suitable for ECG measurements. For EEG measurements which typically make use of relatively high sensitivity, resistor 153 may, for example, have a resistance up to 50 GΩ. In some embodiments, resistor 153 may be tuned (e.g. its resistance value may be varied) in real-time or during a calibration phase. In some embodiments, as shown in FIG. 7, resistor 153 may have a resistance value of 1 GΩ.

In prior art embodiments, a suitable resistor, like resistor 153 is typically configured to directly electrically couple a reference voltage (e.g. typically set at ½ of the power supply voltage 170) to non-inverting input 152+ of first amplifier 152 to thereby statically bias non-inverting input 152+ at this DC voltage level. However, this approach (merely statically setting the DC bias at input 152+), in combination with one or more other factors, such as, for example, internal voltage drifting of amplifier 152, impingement of electromagnetic interference on sensing layer 124, movement of the subject and/or electrode 120 varying capacitance between sensing layer 124 and the subject, etc., may result in saturation of high input impedance amplifier 152.

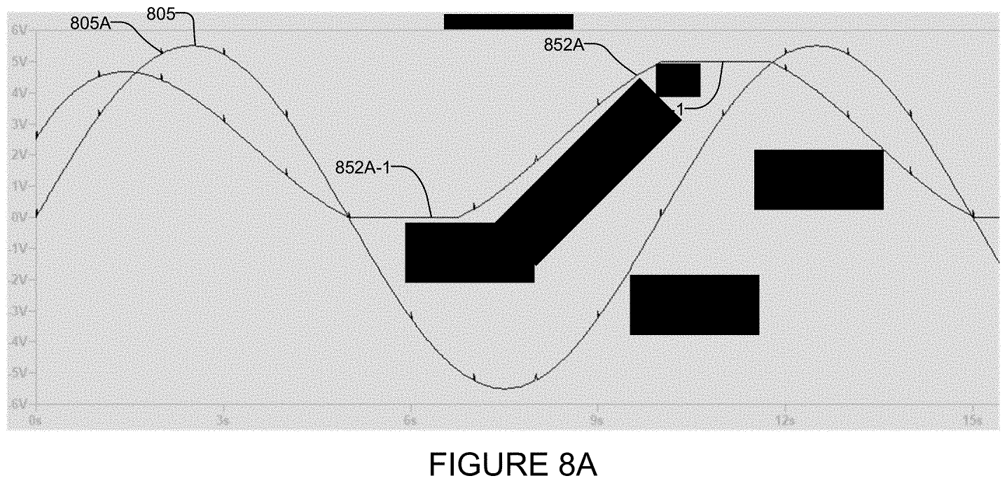

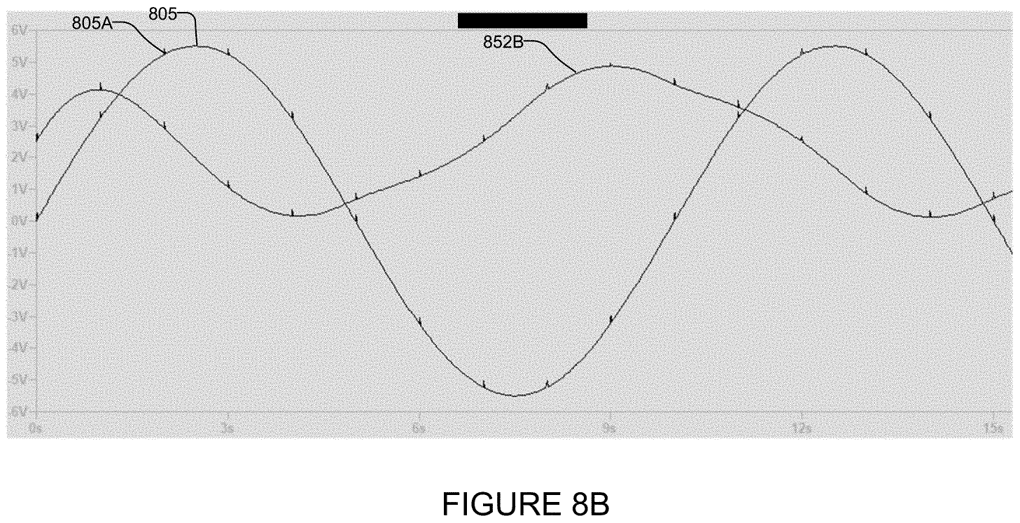

Saturation of amplifier 152 may, for example, result in an inability to faithfully pass sensing signal 105 through amplifier 152 (e.g. see FIG. 8A). Passively biasing amplifier 152 in this manner, allows input amplitude fluctuations at inputs 152+, 152− of amplifier 152 to saturate amplifier 152. By way of none limiting examples, input amplitude fluctuations may be caused by an individual's (e.g. the individual being sensed) movement creating electrostatic charge, movement of other individuals impacting the electromagnetic fields, drift of a direct current (DC) operating voltage of amplifier 152 as a result of operating temperature drifts, movement of the DC operating voltage as a result of low frequency and/or DC electric fields and/or the like. If amplifier 152 is saturated, output signal 152A of amplifier 152 is electrically driven to (and clipped at) either zero volts or the level of the DC supply voltage 170, thereby impeding sensing signal 105 from being passed through amplifier 152. In some embodiments, 20 to 30 seconds may elapse prior to amplifier 152 settling back to normal (i.e. steady state) operating conditions resulting in a loss of (i.e. inability to pass through) sensing signal 105 during that time.

As an alternative to passively electrically coupling resistor 153 to power supply level 170 or reference voltage 178, in particular embodiments of the invention, amplifier circuit 140 comprises a biasing integrator circuit 155 (shown in dashed lines in FIG. 7) for generating an output signal 156A that may be electrically coupled to amplifier 152 (e.g. via resistor 153 to non-inverting input 152+). As will be explained in more detail below (see FIGS. 8A, 8B), integrator circuit 155 minimizes or reduces voltage drifts at input 152+ which may cause saturation of amplifier 152. In some embodiments, integrator circuit 155 comprises amplifier 156, capacitor 155A and resistors 155B, 155C as shown in FIG. 7.

Capacitor 155A electrically couples output 156A of amplifier 156 with inverting input 156− of amplifier 156. Resistor 155B electrically couples output 152A of amplifier 152 with inverting input 156− of amplifier 156. In some embodiments, capacitor 155A may, for example, have a capacitance of 1 μF and resistor 1556 may, for example, have a resistance of 887 kΩ. Varying capacitance of capacitor 155A and/or resistance of resistor 1556 varies a frequency response of integrator circuit 155. For example, larger capacitance and resistance values of capacitor 155A and resistor 155B respectively will slow the frequency response of integrator circuit 155. Conversely, smaller capacitance and resistance values of capacitor 155A and resistor 155B respectively will speed up the frequency response of integrator circuit 155. Reference voltage 178 is electrically coupled to non-inverting input 156+ of amplifier 156 using resistor 155C. Resistor 155C limits input current supplied to non-inverting input 156+ of amplifier 156. In some embodiments, resistor 155C may have a resistance in the range of 1 k Ω to 1M Ω

Integrator circuit 155 continuously monitors signal 152A of amplifier 152 for any detectable voltage drift in signal 152A relative to reference voltage 178. In the event of voltage drift in signal 152A, this drift is reflected at inverting input 156− of amplifier 156, such that output signal 156A of amplifier 156 varies, in an opposite direction, to the detected drift. Electrically coupling output signal 156A to non-inverting input 152+ of amplifier 152 via resistor 153 may, in turn, bias the DC voltage of input 152+ and output signal 152A of amplifier 152 to reference voltage 178.

A time constant of integrator circuit 155 (e.g. response rate of integrator circuit 155 to voltage drifts of signal 152A) may be determined by capacitance of capacitor 155A in combination with resistance of resistor 155B. Varying capacitance of capacitor 155A and/or resistance of resistor 155B varies the time constant of integrator 155. In some embodiments, the time constant may, for example, be varied on a use-specific basis (e.g. one time constant for ECG measurements, a second different time constant for EEG measurements, etc.). In some embodiments, capacitance of capacitor 155A and/or resistance of resistor 1556 may be tuned (i.e. varied) in real time.

In preferred embodiments, sensing signal 105 passes through amplifier 152 unaffected by the effect of integrator circuit 155 supressing voltage drifts of amplifier 152. The time constant of integrator circuit 155 can be set so that integrator circuit 155 is sensitive to relatively slow moving “drifts” of the signal at input 152+ and is relatively insensitive to fast changes in this signal (e.g. see pulses 805A in FIGS. 8A and 8B), which may be associated with sensing signal 105.

In some embodiments, as shown in FIG. 7A, electrode 120 comprises an electrically conductive feedback ring 121. In such embodiments, electrically conductive feedback ring 121 peripherally encloses sensing layer 124. An insulator electrically insulates sensing layer 124 from feedback ring 121. In the illustrated embodiment of FIG. 7A, output signal 156A of integrator circuit 155 may be electrically coupled to feedback ring 121. Electrically coupling output signal 156A to feedback ring 121 generates an electric field between sensing layer 124 and feedback ring 121, resulting in a steady state of output signal 152A being maintained at reference voltage 178. The electric field between sensing layer 124 and feedback ring 121 may be reflected in sensing signal 105 from electrode 120, thereby ensuring that the steady state of output signal 152A is maintained at reference voltage 178. Feedback ring 121 may also apply a constant electric field to an individual's body at the measurement point thereby reducing unwanted electric field changes received by sensing layer 124 and maintaining a more stable input to amplifier 152. Advantageously, the FIG. 7A embodiment may result in amplifier circuit 140 having its highest possible impedance value (i.e. an impedance equivalent to the input impedance value of amplifier 152). In such embodiments, amplifier circuit 140 does not use resistor 153 described elsewhere herein and illustrated, for example, in FIG. 7. Removing resistor 153 may advantageously save PCB space and/or reduce cost of amplifier circuit 140.

In some embodiments, amplifier circuit 140 comprises a digital implementation of integrator 155 as shown in FIG. 7B. Such a digital implementation may comprise, for example, an analog to digital converter (ADC) 192, a processor 194 and a digital to analog converter (DAC) 196. In the FIG. 7B embodiment, ADC 192 samples and digitizes (i.e. produces a digital signal of a corresponding analog signal) output signal 152A or signal 190 (i.e. a filtered signal corresponding to output signal 152A). Processor 194 then processes the generated digital signal. Such digital processing may, for example, mirror the analog processing performed by integrator circuit 155 discussed herein. In some embodiments, processor 194 digitally varies a time constant corresponding to the performed integration in order to maximize rejection of any voltage drifts and/or artifacts that may be present in output signal 152A. DAC 196 receives a digital output from processor 196 and generates a corresponding analog output signal 198 to be electrically coupled with resistor 153 and/or non-inverting input 152+ of amplifier 152. In some embodiments, capacitor 193 and resistor 191 provide an anti-aliasing filter for filtering output signal 152A. Capacitor 193 may, for example, have a capacitance of 100 nF and resistor 191 may, for example, have a resistance value of 100KΩ. In some embodiments, capacitor 199 and resistor 197 provide an anti-aliasing filter for filtering analog output signal 198. Capacitor 199 may, for example, have a capacitance of 100 nF and resistor 197 may, for example, have a resistance value of 100KΩ. A frequency response corresponding to each of the anti-aliasing filters may be varied by varying each filters' capacitance and/or resistance values. In some embodiments, not shown, the digital integrator implementation of the FIG. 7B embodiment may be implemented with the feedback ring embodiment shown in FIG. 7A.

In some embodiments, integrator circuit 155 may be implemented in one or more circuits separate from amplifier circuit 140 as shown in FIG. 7C. For example, integrator circuit 155 may be implemented using a central processor configured (not shown) to receive and process amplified signals 160 in combination with a pre-processor ADC (not shown) and a post-processor DAC (not shown). In preferred embodiments, the central processor is configured to process amplified signals 160 by, for example, filtering amplified signals 160, removing DC components from amplified signals 160, removing artifacts from amplified signals 160 and/or the like. Signal 198A electrically couples amplifier circuit 140 with the post-processor DAC. In some embodiments, a digital link electrically couples amplifier circuit 140 with the central processor. In such embodiments, amplifier circuit 140 comprises a DAC for receiving a digital signal from the central processor and converting the received digital signal to an analog signal (i.e. signal 198A) coupled to amplifier circuit 140. As described elsewhere herein, the combination of resistor 197 and capacitor 199 may provide an anti-aliasing and/or de-noising filter.

FIG. 7D schematically shows an alternative embodiment of amplifier circuit 140. In the example configuration shown in FIG. 7D, amplifier circuit 140 comprises a transimpedence amplifier circuit 300 connected in series with an inverting amplifier 310 to receive and amplify sensing signal 105. This configuration may advantageously minimize the sensitivity of amplifier circuit 140 to changes in the capacitance between sensor 120 and the skin of a subject caused by large and/or slow motion artifacts.

Transimpedence amplifier circuit 300 comprises a feedback capacitor 302 and a feedback resistor 303 connected in parallel with the inverted input 152- and output of amplifier 152. Transimpedence amplifier circuit 300 receives sensing signal 105 from a sensing layer 124 of electrode 120 at the inverted input 152− of amplifier 152. Transimpedence amplifier circuit 300 receives reference voltage 178 at the non-inverted input 152+ of amplifier 152. Transimpedence amplifier circuit 300 outputs an amplified signal 300A.

Feedback capacitor 302 may advantageously help cut off unwanted high frequency noise in sensing signal 105. Feedback capacitor 302 may advantageously stabilize amplifier 152 by compensating for the effect of a low pass-filter formed by the capacitance of sensor 120 and feedback resistor 303. Feedback capacitor 302 may have capacitances which are typically in the range of 1-100 μF, although other capacitance values are possible. Feedback capacitor 302 may have capacitances which are tuned based on the resistance of feedback resistor 303.

Feedback resistor 303 may be tuned to control the gain of transimpedence amplifier circuit 300. In some embodiments, feedback resistor 303 is a variable resistor (e.g. a trimmer resistor, a potentiometer, etc.) having a resistance that is adjustable between values which are typically in the range of 1MΩ-500MΩ. In some embodiments, this range of adjustability may be larger (e.g. 500 kΩ-1 GΩ) or smaller.

In some embodiments, the output of transimpedence amplifier circuit 300 is connected to an inverting amplifier 310. In the example embodiment shown in FIG. 7D, inverting amplifier 310 receives amplified signal 300A at the inverted input 154− of amplifier 154. Inverting amplifier 310 receives reference voltage 178 at the non-inverted input 154+ of amplifier 154. Inverting amplifier 310 outputs an output signal 310A. Output signal 310A is transmitted to a measuring system (e.g. base unit 180) for further signal processing.

Inverting amplifier 310 advantageously acts a buffer for transimpedence amplifier circuit 300 by providing high input impendence and low output impendence. Inverting amplifier 310 is a unity gain inverting amplifier in the FIG. 7D example embodiment, but this is not necessary. Inverting amplifier 310 may comprise suitable resistors and/or capacitors to further adjust the gain and/or frequency response of inverting amplifier 310. This FIG. 7D approach may minimize the sensitivity of the amplifier to changes in the capacitance between the sensor and the subject's body (e.g. which may be a result of movements of the person). This change in capacitance can create motion artifact voltages in a voltage-mode amplifier. The current-mode amplifier of the FIG. 7D example may thus be less sensitive to this effect.

In some embodiments, reference voltage 178 is generated from a reference source 400 (see FIG. 7D). Reference source 400 may comprises voltage dividers or the like to convert power supply voltage 170 into reference voltage 178. In some embodiments, reference source 400 has a high output impedance.

In some embodiments, amplifiers 154, 156 and/or 158 may, for example, be high input impedance and/or low noise operational amplifiers. In some embodiments, amplifiers 154, 156 and/or 158 may, for example, be high input impedance operational amplifiers manufactured by Texas Instruments of Dallas, Tex. under part number LMP7715 or the like. In some embodiments, amplifier 152 has a higher input impedance than amplifiers 154, 156 and/or 158.

Electrical leads (not explicitly shown) may, for example, electrically couple non-inverting input 152+ of amplifier 152, buffer signal 159 and ground signal 172 respectively to contact plates 142A within port 142 of housing 141.

In some embodiments, power supply voltage 170 and ground signal 172 may be electrically coupled to positive and negative electrical power inputs respectively of amplifiers 152, 154, 156 and/or 158. This connection is omitted in FIG. 7 for clarity. In some embodiments, one or more capacitors may be coupled across positive and negative electrical power inputs of one or more amplifiers 152, 154, 156 and/or 158 for reducing an amount of electromagnetic interference present across the power inputs of amplifiers 152, 154, 156 and/or 158. In some embodiments, capacitors may be coupled across reference voltage 178 and ground signal 172 for reducing an amount of electromagnetic interference present in reference voltage 178.

In some embodiments, reference voltage 178 is equivalent to half of power supply voltage 170. In some embodiments, power supply voltage 170 and reference voltage 178 are equivalent to 5 and 2.5 Volts DC.

In some embodiments, amplifier circuit 140 may be electrically assembled on a single printed circuit board (PCB) 140A. In other embodiments, amplifier circuit 140 may, for example, be electrically assembled using a plurality of electrically coupled PCBs 140A. PCB(s) 140A may be housed within housing 141 of contactless electrode system 100 as described herein.

FIGS. 8A-B depict simulation results observed by the inventors corresponding to an expected signal response behavior of amplifier 152 in FIGS. 7-7C. In the FIGS. 8A-B examples, curve 805 corresponds to biopotentials measurable by electrode 120. Curve 805 is set as a low frequency (e.g 0.1 Hz in the FIGS. 8A-B example) sine wave to represent large motion artifacts (e.g. human physiological and/or natural movements). Curve 805 comprises a series of relatively high frequency (e.g. 1 Hz in the FIGS. 8A-B example) pulses 805A which represent a heartbeat of the individual.

In the FIG. 8A example, curve 852A corresponds to an output signal 152A of amplifier 152 in a circuit (not shown) which does not feed output signal 152A back to non-inverting input 152+ through an integrator 155. Large voltage drifts represented by the large amplitude of curve 805 causes output signal 152A to clip at either zero volts or DC supply voltage 170 (e.g. 5V) which is represented by the clipped portions 852A-1 of curve 852A. In the FIG. 8A example, about three seconds elapses prior to amplifier 152 settling back to normal operating conditions resulting in an inability for amplifier 152 to detect pulses 805A during that time.

In the FIG. 8B example, curve 852B corresponds to an output signal 152A of amplifier 152 in a circuit (e.g. amplifier circuit 140) which feeds output signal 152A back to non-inverting input 152+ through an integrator 155 (e.g. see FIG. 7). Integrator 155 advantageously biases the DC voltage of input 152+ in a direction opposite of the direction of the voltage drift to prevent output signal 152A from clipping at either zero volts or DC supply voltage 170 (e.g. 5V). As can be seen in the FIG. 8B example, curve 852B which represents an output signal 152A of amplifier 152 in amplifier circuit 140 does not clip at zero volts and/or DC supply voltage 170 thereby allowing amplifier 152 to detect pulses continuously.

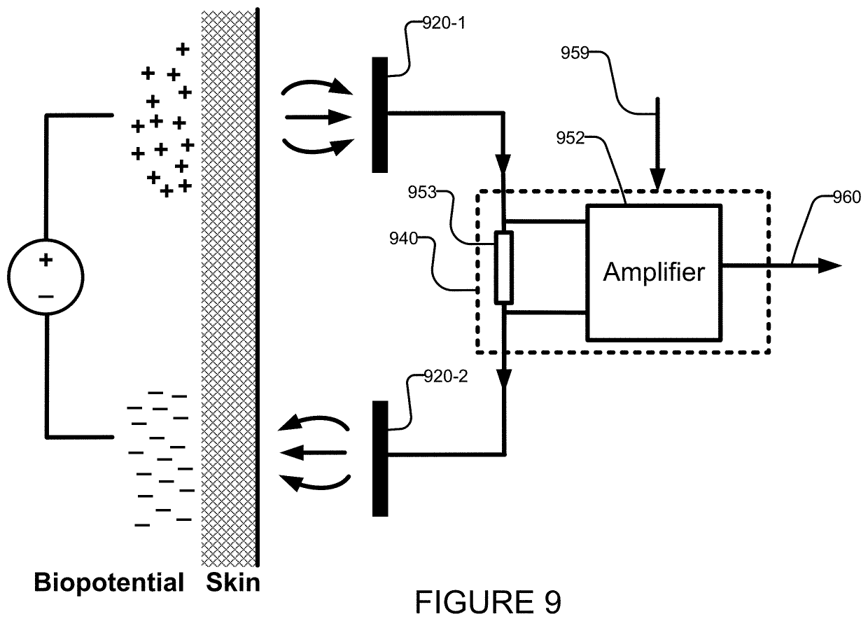

FIG. 9 schematically shows a current sensing amplifier circuit 940 suitable for use with two contactless electrodes 920-1, 920-2 according to an example embodiment. Electrodes 920-1, 920-2 may comprise any electrode suitable for detecting biopotentials (e.g. contactless electrodes 120).

In the example embodiment shown in FIG. 9, current sense amplifier circuit 940 comprises a current sense amplifier 952 and a sense resistor 953. Current sense amplifier 952 may have high input impedance. Sense resistor 953 is electrically coupled to a first contactless electrode 920-1 and a second contactless electrode 920-2. First contactless electrode 920-1 forms a first skin-electrode capacitor at a first location along an individual's skin. Second contactless electrode 920-2 forms a second skin-electrode capacitor at a second location along an individual's skin. Sense resistor 953 has resistance values which are typically in the range of 1-10MΩ.

Biopotentials can create various temporary electric fields near contactless electrodes 920-1, 920-2. These electric fields may induce current flow across sense resistor 953. In the example embodiment shown in FIG. 9, positive charges at the first location of the individual's skin attracts negative charges in first contactless electrode 920-1 causing positive charges in first contactless electrode 920-1 to be repelled away from the individual's skin at the first location. Negative charges at the second location of the individual's skin attracts positive charges in second contactless electrode 920-2 causing negative charges in second contactless electrode 920-2 to be repelled away from the individual's skin at the second location. In the example embodiment shown in FIG. 9, an induced current is generated as a result of electrons flowing from second contactless electrode 920-2 to first contactless electrode 920-1.

Current sense amplifier 952 is electrically coupled across sense resistor 953 to convert and/or amplify current flow across sense resistor 953 to an output signal 960. In some embodiments, current sense amplifier 952 may, for example, be high-side current sense amplifiers manufactured by Analog Devices of Milpitas, Calif. under part number LT6100 or the like.

In some embodiments, current sensing amplifier circuit 940 comprises an additional gain stage (not shown) which amplifies output signal 960. In some embodiments, current sensing amplifier circuit 940 receives a gain control signal 959. Gain control signal 959 may provide automatic gain control to current sense amplifier 952 through additional suitable circuitry (not shown).

Sensing amplifier circuit 940 is advantageously inherently resistant to common mode signals (i.e. signals that induce the same instantaneous electrical potential and phase at contactless electrodes 920) since such signals do not generate current flow across sense resistor 953. Sensing amplifier circuit 940 advantageously mitigates saturation issues caused by static electrical fields since current induced by static fields will zero out in steady state. Sensing amplifier circuit 940 advantageously mitigates microphonic effects caused by large motion artifacts since noise generated at electrodes 920-1 and 920-2 will cancel out.

FIG. 10 schematically illustrates a biopotential measurement system 300 according to a particular embodiment. In some embodiments, biopotential measurement system 300 comprises a plurality (e.g. a pair in the illustrated embodiment) of electrode systems 200-1, 200-2 (e.g. see FIG. 6C) which may be used, for example, to measure a single-lead ECG. In such embodiments, contactless electrodes 220-1, 220-2 may be capacitively coupled to an individual's right and left arms respectively. Each of electrode systems 200-1, 200-2 may be similar to the electrode systems described elsewhere herein. Amplified signals 260-1, 260-2 may be transmitted to base unit 280 (which may be similar to base unit 180 described elsewhere herein) using cables 210-1, 210-2 respectively (not explicitly shown). In some embodiments, amplified signals 260-1, 260-2 are transmitted to base unit 280 using a suitable wireless communication interface. ECG processor 284 may, for example, amplify a difference between amplified signals 260-1, 260-2 generating and/or displaying (using, for example, optional display 286) a lead-I ECG. Power supply 282 of base unit 280, for example, generates power signal 270 provided to electrode systems 200-1, 200-2 using cables 210-1, 210-2 respectively. In another embodiment, biopotential measurement system 300 may comprise three contactless electrode systems 200-1, 200-2, 200-3 (not explicitly shown). A standard 3-lead ECG may be measured, for example, by capacitively coupling contactless electrodes 220-1, 220-2 and 220-3 (not explicitly shown) to an individual's right arm (RA), left arm (LA) and left leg (LL) respectively. In another embodiment of bio-potential measurement system 300, an EEG may be measured by, for example, further increasing a number of contactless electrode systems 200-1 . . . 200-n used by bio-potential measurement system 300. Electrode systems 200-1 . . . 200-n may be functionally equivalent to electrode systems 100 described herein.

In some embodiments, ECG processor 284 generates a RLD (i.e. Right Leg Drive) signal 261 which may be fed back to a patient's body. RLD signal 261 may be generated from a combination of sensor signals (e.g. amplified signals 260-1, 260-2). RLD signal 261 may be inverted to be opposite in phase compared to the sensor signals. In some embodiments, RLD signal 261 is generated by averaging multiple amplified signals (e.g. amplified signals 260-1, 260-2) and subsequently inverting the phase of the averaged signal. In a currently preferred embodiment, RLD signal 261 is fed back to a patient's body through capacitive coupling (i.e. a non-contact sensor is placed proximate to the patient's skin) although other methods (e.g. contact based methods) for feeding RLD signal 261 back to a patient's body are possible. Feeding RLD signal 261 back to a patient's body can advantageously help suppress common mode noise caused by, for example, line interference and/or the like.

Example Use Cases and Applications

In some embodiments, contactless electrode system 100 described herein may be implemented in a vehicular setting (e.g. inside a car, truck, bus, plane, boat or the like). Such embodiments may comprise embedding one or more of contactless electrode systems 100 or one or more of electrodes 120 into components of the vehicle, such as (without limitation): the vehicle seat(s), seat restraints, the steering wheel, the dashboard, the vehicle ceiling, the vehicle floor and/or the like. Embedded contactless electrode systems 100 or electrodes 120 may, for example, be used to determine the state of an individual's heart muscle (i.e. ECG measurement) and/or the skeletal or other muscle (i.e. EMG measurement) of the vehicle operator. Such information may be communicated to first responders or suitable authorities in the event of an accident or during normal vehicular operation periods. Such embodiments can also alert a vehicle operator (e.g. using suitable alarms or the like) that the vehicle operator is having a cardiac event (e.g. a heart attack) or similar heart condition. Data from such vehicular ECG systems and/or EMG systems may be recorded—e.g. for forensic analysis, data analytics or the like. In some embodiments, data from such vehicular ECG systems and/or EMG systems may be used to adjust the vehicle seat(s), steering wheel, seat warmer(s), seat vent(s), air conditioning settings, or the like. In some embodiments, different emotional states (e.g. a stressed state, a relaxed state, etc.) detected using such data may trigger different adjustments (e.g. a vehicle seat may be adjusted differently depending on a detected emotional state, air-conditioning settings may be set to different temperatures depending on whether an individual is in a stressed state or a relaxed state, etc.).

In an example implementation of contactless electrode system 100 in a vehicular setting, contactless electrode system 100 measures various biopotentials of an individual seated inside of a vehicle to calculate a heart rate variability (i.e. variation in the time interval between heartbeats) of the individual. Since electrodes 120 can advantageously detect biopotentials without making contact with the individual, contactless electrode system 100 may be embedded in the vehicle seat(s), seat restraints, steering wheel, dashboard, vehicle ceiling, vehicle floor, etc. Contactless electrode system 100 in the example use case describe herein is coupled to a computer (e.g. ECU) in the vehicle. The computer may monitor the heart rate variability of individuals in a vehicle in real time. The computer may determine that a person is likely too hot or too cold based on their heart rate and/or heart rate variability. The computer may adjust the temperature inside of the vehicle (i.e. adjust the AC system inside the vehicle) based on this determination (i.e. based on the heart rate and/or heart rate variability of the individual).

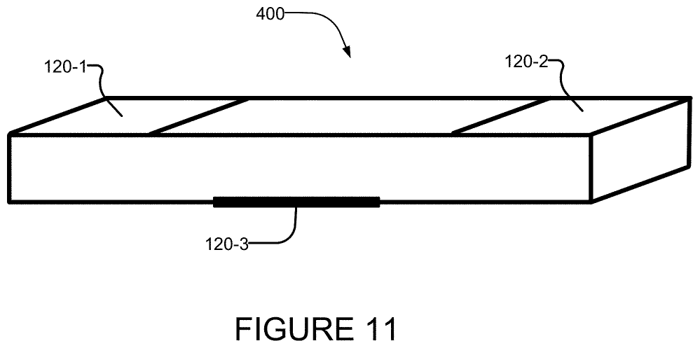

In some embodiments, contactless electrode system 100 described herein may be implemented as a portable device (e.g. a phone, a table, a computer, a standalone portable device, etc.). Such embodiments may comprise embedding one or more of contactless electrode systems 100 or one or more of electrodes 120 in different locations of the portable device. In the example embodiment shown in FIG. 11, portable device 400 comprises electrodes 120-1, 120-2, 120-3 embedded in opposing faces of portable device 400. Portable device 400 comprises a LA electrode 120-1 located on a left side of a first face of portable device 400, a RA electrode 120-2 located on a right side of the first face of portable device 400, and a LL electrode 120-3 located in the middle of a second opposing face of portable device 400. Portable device 400, as illustrated in the FIG. 11 embodiment, comprises three contactless electrodes 120, but this is not necessary. In other embodiments, portable device 400 may comprise one or more contact electrodes in addition to or in alternative to contact electrodes 120. For example, portable device 400 may comprise a contact LA electrode 120-1, a contact RA electrode 120-2, and a contactless LL electrode 120-3. With this embodiment, an individual may use portable device 400 to determine the state of the individual's heart muscle by touching LA electrode 120-1 with a left upper part of the individual's body (e.g. left hand, left arm, etc.), touching RA electrode 120-2 with a right upper part of the individual's body (e.g. right hand, right arm, etc.), and positioning contactless LL electrode 120-3 in proximity to a left lower part of the individual's body (e.g. left leg, left ankle, left foot, etc.). Portable device 400 may comprise one or more amplifier circuits 140 as described elsewhere herein. Portable device 400 may transmit data wirelessly to a base unit 180, 280 (e.g. a phone, a computer, a smartwatch, etc.) as described elsewhere herein.

In some embodiments, amplified signals 160 corresponding to, for example, ECG measurements may, for example, be analyzed to determine respiration rates and/or respiration patterns of an individual. In such embodiments, the respiration information may be used alone or in conjunction with ECG data or other data (e.g. EEG data, EMG data or EOG data) to determine a state of an individual, such as, for example, whether the individual is asleep, drowsy, impaired, is suffering from medical conditions or the like.

In some embodiments, amplified signals 160 may be analyzed alone or in combination with other signals to determine a medical state of an individual and/or provide analytics related to, for example, drowsiness, unconsciousness, incapacity, brain injury, stroke, arrhythmias, compensated shock, decompensated shock, sepsis, heart attack, sleep apnea, stress, attentiveness, cognition, respirations, internal bleeding, body temperature, personal identification, electrolyte imbalance, or the like.

In some embodiments, amplified signals 160 may be analyzed alone or in combination with other signals to identify an individual. For example, an amplified signal 160 may be compared against one or more known signals (ECG signals, EEG signals, EMG signals, EOG signals, etc.), each signal representative of a different individual's identity. In some embodiments, amplified signal 160 is an ECG signal. In such embodiments, differences in parameters such as resting heart rates, QRS complexes, etc. may, for example, be used to match amplified signal 160 to (or differentiate amplified signal 160 from) one or more ECG signals representative of different identities.

In some embodiments, a vehicle embedded system as described elsewhere herein may ascertain the identities of the vehicle operator and/or passenger(s). Upon ascertaining the identities, the vehicle may, for example, automatically adjust the vehicle seat(s), steering wheel, environmental conditions or the like according to each of the identified individual's pre-configured preferences.

In some embodiments, software may be used to interpret amplified signals 160 to provide detailed information about the state of an individual.

In some embodiments, one or more of contactless electrode systems 100 may be incorporated or embedded into electronic devices such as, for example, cellular phones, tablets, laptop computers, desktop computers, smart watches, activity trackers, etc. In some embodiments, one or more of contactless electrode systems 100 may be incorporated or embedded into animal vests, animal beds, infant hospital beds, infant incubators, clothing or the like and/or casing or other protective gear for such devices. In some embodiments, one or more of contactless electrode systems 100 may be incorporated or embedded into, for example, hospital beds, gurneys, wheel-chairs, medical examination tables, household furnishings including household bed frames or the like.

In some embodiments, one or more contactless electrode systems 100 may be incorporated or embedded in a headwear (e.g. helmets, caps, etc.). In such embodiments, contactless electrode system 100 may measure EEG (from the head) instead of or in addition to ECG. In some embodiments, EEG and ECG apparatus may be configured to operate on an individual. The computer may switch between ECG and EEG operation or may perform both simultaneously.

In some embodiments, contactless electrode system 100 comprises a Global Positioning System (GPS) locator which continuously tracks the location of contactless electrode system 100.

In some embodiments, the systems and methods described herein are not limited to humans and may be used for measurement of electrical activity within animals, such as, for example, pet animals, zoo animals, rescued wild animals, wild animals or the like. Accordingly, unless the context clearly requires otherwise, throughout the description and the claims, “individual” is to be construed as inclusive of both human subjects as well as animal subjects.

In some embodiments, where amplified signals 160 capture signals related to the operation of cell(s), tissue(s), organ(s) and/or system(s), base unit 180 may be configured to use these signals (individually and/or together) to create and display animation on a suitable display. The displayed animation may be based on one or more amplified signals 160 and may, for example, show the operation of the cell(s), tissue(s), organ(s) and/or system(s).

Interpretation of Terms

Unless the context clearly requires otherwise, throughout the description and the claims:
“comprise”, “comprising”, and the like are to be construed in an inclusive sense, as opposed to an exclusive or exhaustive sense; that is to say, in the sense of “including, but not limited to”;
“connected”, “coupled”, or any variant thereof, means any connection or coupling, either direct or indirect, between two or more elements; the coupling or connection between the elements can be physical, logical, or a combination thereof; elements which are integrally formed may be considered to be connected or coupled;
“herein”, “above”, “below”, and words of similar import, when used to describe this specification, shall refer to this specification as a whole, and not to any particular portions of this specification;
“or”, in reference to a list of two or more items, covers all of the following interpretations of the word: any of the items in the list, all of the items in the list, and any combination of the items in the list; and
the singular forms “a”, “an”, and “the” also include the meaning of any appropriate plural forms.

Words that indicate directions such as “vertical”, “transverse”, “horizontal”, “upward”, “downward”, “forward”, “backward”, “inward”, “outward”, “vertical”, “transverse”, “left”, “right”, “front”, “back”, “top”, “bottom”, “below”, “above”, “under”, and the like, used in this description and any accompanying claims (where present), depend on the specific orientation of the apparatus described and illustrated. The subject matter described herein may assume various alternative orientations. Accordingly, these directional terms are not strictly defined and should not be interpreted narrowly.

Embodiments of the invention may be implemented using specifically designed hardware, configurable hardware, programmable data processors configured by the provision of software (which may optionally comprise “firmware”) capable of executing on the data processors, special purpose computers or data processors that are specifically programmed, configured, or constructed to perform one or more steps in a method as explained in detail herein and/or combinations of two or more of these. Examples of specifically designed hardware are: logic circuits, application-specific integrated circuits (“ASICs”), large scale integrated circuits (“LSIs”), very large scale integrated circuits (“VLSIs”), and the like. Examples of configurable hardware are: one or more programmable logic devices such as programmable array logic (“PALs”), programmable logic arrays (“PLAs”), and field programmable gate arrays (“FPGAs”)). Examples of programmable data processors are: microprocessors, microcontrollers, digital signal processors (“DSPs”), embedded processors, graphics processors, math co-processors, general purpose computers, server computers, cloud computers, mainframe computers, computer workstations, and the like. For example, one or more data processors in a computer system for a device may implement methods as described herein by executing software instructions in a program memory accessible to the processors.

Processing may be centralized or distributed. Where processing is distributed, information including software and/or data may be kept centrally or distributed. Such information may be exchanged between different functional units by way of a communications network, such as a Local Area Network (LAN), Wide Area Network (WAN), or the Internet, wired or wireless data links, electromagnetic signals, or other data communication channel.

For example, while processes or blocks are presented in a given order, alternative examples may perform routines having steps, or employ systems having blocks, in a different order, and some processes or blocks may be deleted, moved, added, subdivided, combined, and/or modified to provide alternative or subcombinations. Each of these processes or blocks may be implemented in a variety of different ways. Also, while processes or blocks are at times shown as being performed in series, these processes or blocks may instead be performed in parallel, or may be performed at different times.

In addition, while elements are at times shown as being performed sequentially, they may instead be performed simultaneously or in different sequences. It is therefore intended that the following claims are interpreted to include all such variations as are within their intended scope.

Embodiments of the invention may also be provided in the form of a program product. The program product may comprise any non-transitory medium which carries a set of computer-readable instructions which, when executed by a data processor, cause the data processor to execute a method of the invention. Program products according to the invention may be in any of a wide variety of forms. The program product may comprise, for example, non-transitory media such as magnetic data storage media including floppy diskettes, hard disk drives, optical data storage media including CD ROMs, DVDs, electronic data storage media including ROMs, flash RAM, EPROMs, hardwired or preprogrammed chips (e.g. EEPROM semiconductor chips), nanotechnology memory, or the like. The computer-readable signals on the program product may optionally be compressed or encrypted.

In some embodiments, the invention may be implemented in software. For greater clarity, “software” includes any instructions executed on a processor, and may include (but is not limited to) firmware, resident software, microcode, and the like. Both processing hardware and software may be centralized or distributed (or a combination thereof), in whole or in part, as known to those skilled in the art. For example, software and other modules may be accessible via local memory, via a network, via a browser or other application in a distributed computing context, or via other means suitable for the purposes described above.

Where a component (e.g. a software module, processor, assembly, device, circuit, etc.) is referred to above, unless otherwise indicated, reference to that component (including a reference to a “means”) should be interpreted as including as equivalents of that component any component which performs the function of the described component (i.e. that is functionally equivalent), including components which are not structurally equivalent to the disclosed structure which performs the function in the illustrated exemplary embodiments of the invention.

Where a record, field, entry, and/or other element of a database is referred to above, unless otherwise indicated, such reference should be interpreted as including a plurality of records, fields, entries, and/or other elements, as appropriate. Such reference should also be interpreted as including a portion of one or more records, fields, entries, and/or other elements, as appropriate. For example, a plurality of “physical” records in a database (i.e. records encoded in the database's structure) may be regarded as one “logical” record for the purpose of the description above and the claims below, even if the plurality of physical records includes information which is excluded from the logical record.

Specific examples of systems, methods and apparatus have been described herein for purposes of illustration. These are only examples. The technology provided herein can be applied to systems other than the example systems described above. Many alterations, modifications, additions, omissions, and permutations are possible within the practice of this invention. This invention includes variations on described embodiments that would be apparent to the skilled addressee, including variations obtained by: replacing features, elements and/or acts with equivalent features, elements and/or acts; mixing and matching of features, elements and/or acts from different embodiments; combining features, elements and/or acts from embodiments as described herein with features, elements and/or acts of other technology; and/or omitting combining features, elements and/or acts from described embodiments.

It is therefore intended that the following appended claims and claims hereafter introduced are interpreted to include all such modifications, permutations, additions, omissions, and sub-combinations as may reasonably be inferred. The scope of the claims should not be limited by the preferred embodiments set forth in the examples, but should be given the broadest interpretation consistent with the description as a whole.

What is claimed is:

1. A contactless system for sensing biopotentials in an individual, the system comprising:
an electrode for generating a sensing signal indicative of a biopotential at a location on a body of the individual, the electrode comprising:
an electrically conductive sensing layer having a sensing surface, an opposing surface which opposes the sensing surface and one or more edge surfaces extending between the sensing surface and the opposing surface, the sensing surface capacitively coupled to an outer tissue surface of the individual and sensitive to electric field in a vicinity of the sensing surface; and
an electrically conductive guard layer proximate to the opposing surface of the sensing layer and separated from the opposing surface by an electrically non-conductive layer, the guard layer electrically insulating the sensing layer from electromagnetic interference; and
a high input impedance amplifier circuit
wherein the sensing layer is electrically coupled to an input of the high input impedance amplifier circuit to condition the sensing signal into an amplifier output signal that depends at least in part on capacitive coupling between the sensing layer and the tissue surface of the individual.

2. A system according to claim 1 wherein the guard layer is electrically coupled to a buffer amplifier of the high input impedance amplifier circuit to receive a buffer signal, the buffer signal comprising an amplitude and a phase corresponding to an amplitude and a phase of the sensing signal.

3. A system according to claim 1 wherein the electrode comprises an electrically conductive guard ring peripherally enclosing the sensing layer, the guard ring electrically insulating the sensing layer from electromagnetic interference from electromagnetic energy that impinges on the guard ring.

4. A system according to claim 3 wherein an inner edge surface of the guard ring is separated from an outer edge surface of the sensing layer by an electrically non-conductive ring.

5. A system according to claim 3 wherein the guard ring is electrically coupled to a buffer amplifier of the high input impedance amplifier circuit to receive a buffer signal, the buffer signal comprising an amplitude and a phase corresponding to an amplitude and a phase of the sensing signal.

6. A system according to claim 2 wherein the buffer signal comprises an amplitude and a phase substantially identical to the amplitude and the phase of the sensing signal.

7. A system according to claim 1 wherein the electrode comprises an electrically conductive grounding layer proximate to an upper surface of the guard layer and separated from the upper surface of the grounding layer by an electrically non-conductive layer, the grounding layer electrically insulating the electrode from electromagnetic energy, the grounding layer electrically coupled to an electrical ground signal of the high input impedance amplifier circuit.

8. A system according to claim 1 wherein the sensing signal generated by the sensing layer is electrically coupled to a high input impedance amplifier of the high input impedance amplifier circuit and wherein the high input impedance amplifier is configured to generate a high input impedance amplifier output signal.

9. A system according to claim 8 wherein the high input impedance amplifier is a unity gain amplifier.

10. A system according to claim 8 wherein the high input impedance amplifier circuit comprises a biasing integrator circuit connected to provide feedback which maintains a DC component of the high input impedance amplifier output signal within operational voltage limits of the high input impedance amplifier circuit, wherein the biasing integrator circuit is configured to generate a biasing signal which varies in opposition to drift of the DC component relative to a reference voltage, the biasing signal electrically coupled to the input of the high impedance amplifier circuit via a resistor.

11. A system according to claim 10 wherein the electrode comprises a feedback ring, the feedback ring peripherally enclosing the sensing layer, the feedback ring electrically coupled to receive the biasing signal, wherein electrically coupling the feedback ring to the biasing signal maintains the DC component at the reference voltage.

12. A system according to claim 10 wherein the biasing integrator circuit is connected to receive the high input impedance amplifier output signal at an inverting input of an amplifier of the integrator circuit and is configured to integrate the high input impedance amplifier output signal over time to generate the biasing signal.

13. A system according to claim 8 wherein the high input impedance amplifier output signal is electrically coupled to a gain amplifier for generating the amplifier output signal.

14. A system according to claim 13 wherein a high-pass filter is interposed between the high input impedance amplifier and the gain amplifier.

15. A system according to claim 13 wherein the gain amplifier comprises a voltage gain greater than or equal to 10.

16. A system according to claim 13 wherein the gain amplifier comprises a corner lower frequency of 0.72 Hz or less.

17. A system according to claim 13 wherein the gain amplifier comprises a corner high frequency of 338.62 Hz or more.

18. A system according to claim 1 wherein the high input impedance amplifier circuit is housed within a housing, the housing electrically insulating the high input impedance amplifier circuit from electromagnetic interference from electromagnetic energy that impinges on the housing.

19. A system according to claim 18 wherein the housing is hermetically sealed.

20. A system according to claim 18 wherein the housing is waterproof.
